SEQUENCE LISTING (118 sequences)

SEQ ID NO: 1 - DNA, 22 bp, Arabidopsis thaliana
taacccaacaccttacggcacg

SEQ ID NO: 2 - DNA, 18 bp, Arabidopsis thaliana
cggacacaggtggtgcat

SEQ ID NO: 3 - DNA, 18 bp, Arabidopsis thaliana
tgttgatgtttataccga

SEQ ID NO: 4 - DNA, 19 bp, Arabidopsis thaliana
tcggaatgaatcacaaaat

SEQ ID NO: 5 - DNA, 16 bp, Arabidopsis thaliana
tttcgcgtctctctaa

SEQ ID NO: 6 - DNA, 19 bp, Arabidopsis thaliana
ttaaataaaccaaggattt

SEQ ID NO: 7 - DNA, 549 bp, Artificial Sequence
  Cytidine deaminase half and uracil glycosylase inhibitor
ggtagtccaactccgtatccgaattacgccaatgcaggacatgttgaaggtcaatctgcattgttcatgagggataacggcatttctgaagggttggtgttccacaacaaccctgaaggaacatgtggattttgcgtcaacatgacagaaacccttctcccagaaaacgctaagatgacagtagttccacctgaaggtgctattcctgtcaaaagaggtgctactggtgaaaccaaggtgtttactgggaattccaattcacccaaaagcccaacgaaaggtgggtgtagtggaggatctacaaatctctctgacatcattgagaaagagactggaaagcaactagtcattcaggagtcaatcctgatgttaccagaggaggttgaggaagtgataggcaataagcccgaaagcgatatacttgttcatactgcctatgacgaatcgacggatgagaacgtaatgcttctaacctcagatgctcctgagtacaaaccttgggcgttagttatccaggattccaatggagagaacaagatcaagatgttg

SEQ ID NO: 8 - DNA, 399 bp, Artificial Sequence
  Cytidine deaminase half and uracil glycosylase inhibitor
ggatctggtagctatgcgttaggaccctatcagatttcagctcctcaattgcctgcctataatgggcaaactgttggcaccttttactacgtcaatgatgctggagggttagaatccaaggtgttctcaagtggtggttctggaggtagtacgaatctttcggacatcatagagaaggaaactggaaaacagctcgttatccaagagagcattctcatgttgccagaagaagttgaagaggttataggcaacaaaccggaatctgacattctggtacataccgcttatgatgagtcaacagatgagaacgtcatgcttttgacatctgatgcaccagaatacaaaccttgggcacttgtgattcaggattccaatggtgagaacaagatcaagatgcta

SEQ ID NO: 9 - DNA, 357 bp, Artificial Sequence
  Cytidine deaminase half and uracil glycosylase inhibitor
ggttctgcgattccagttaagagaggagctacaggagaaacgaaagtctttactgggaattccaattctcccaaatcaccgactaaaggcggatgtagtggtggtagtaccaatctttccgacattatcgagaaggaaacaggtaaacaactcgtaatccaagaaagcatactgatgcttcctgaagaggttgaagaggtcatagggaacaaacctgaaagcgacattttggttcatactgcctatgatgagtctacagatgagaacgtgatgttgctaacctcagatgcacctgaatacaagccatgggctttagtgattcaggattcgaatggagagaacaagatcaagatgctc

SEQ ID NO: 10 - DNA, 590 bp, Artificial Sequence
  Cytidine deaminase half and uracil glycosylase inhibitor
gggtctggatcgtatgctttaggaccgtatcagatctcagctccacaattgcctgcatataacggacaaactgttgggaccttttactacgttaacgatgctggtggattggagtccaaagtgttctcttctggtggcccaactccatatcccaattatgcgaatgcaggccatgttgaaggtcaatcagccctattcatgagagataacggaataagtgaaggactggtgtttcacaacaatccagaaggtacttgtggattttgcgtaaacatgactgagacacttctcccagaaaatgccaagatgacagttgtacctcctgaaggttctggtggatcgacaaacctttcagacattatcgagaaagagacaggcaaacagctagtgattcaagagtccattctcatgcttcccgaagaagttgaggaagtcattgggaataagccggaaagtgacatactcgttcatacggcttacgatgagagcacggatgagaatgtcatgttgcttaccagtgatgcacctgaatacaaaccttgggcttagtcatccaggacagcaatggtgagaacaagatcaagatgctg

SEQ ID NO: 11 - DNA, 162 bp, Arabidopsis thaliana
atggattcacagctagtcttgtctctgaagctgaatccaagcttcactcctctttctcctctcttccctttcactccatgttcttctttttcgccgtcgctccggttttcttcttgctactcccgccgcctctattctccggttaccgtctacgccgcgaag

SEQ ID NO: 12 - DNA, 20 bp, Artificial Sequence
  Synthetic primer
tgataactcgagcgatcctc

SEQ ID NO: 13 - DNA, 20 bp, Artificial Sequence
  Synthetic primer
ccccaatccctttttcactg

SEQ ID NO: 14 - DNA, 35 bp, Artificial Sequence
  Synthetic primer
aaaaagggattggggggtagtccaactccgtatcc

SEQ ID NO: 15 - DNA, 39 bp, Artificial Sequence
  Synthetic primer
tcgctcgagttatcacaacatcttgatcttgttctctcc

SEQ ID NO: 16 - DNA, 35 bp, Artificial Sequence
  Synthetic primer
aaaaagggattggggggatctggtagctatgcgtt

SEQ ID NO: 17 - DNA, 39 bp, Artificial Sequence
  Synthetic primer
tcgctcgagttatcatagcatcttgatcttgttctcacc

SEQ ID NO: 18 - DNA, 36 bp, Artificial Sequence
  Synthetic primer
aaaaagggattggggggttctgcgattccagttaag

SEQ ID NO: 19 - DNA, 36 bp, Artificial Sequence
  Synthetic primer
tcgctcgagttatcagagcatcttgatcttgttctc

SEQ ID NO: 20 - DNA, 35 bp, Artificial Sequence
  Synthetic primer
aaaaagggattgggggggtctggatcgtatgcttt

SEQ ID NO: 21 - DNA, 36 bp, Artificial Sequence
  Synthetic primer
tcgctcgagttatcacagcatcttgatcttgttctc

SEQ ID NO: 22 - DNA, 25 bp, Artificial Sequence
  Synthetic primer
atggattcacagctagtcttgtctc

SEQ ID NO: 23 - DNA, 21 bp, Artificial Sequence
  Synthetic primer
cttcgcggcgtagacggtaac

SEQ ID NO: 24 - DNA, 25 bp, Artificial Sequence
  Synthetic primer
atggattcacagctagtcttgtctc

SEQ ID NO: 25 - DNA, 41 bp, Artificial Sequence
  Synthetic primer
gtctacgccgcgaagacaactttgtataataaagttgaacg

SEQ ID NO: 26 - DNA, 36 bp, Artificial Sequence
  Synthetic primer
gtctacgccgcgaaggctagtgatatcacaagtttg

SEQ ID NO: 27 - DNA, 20 bp, Artificial Sequence
  Synthetic primer
ggttccaaactcaacggtgg

SEQ ID NO: 28 - DNA, 20 bp, Artificial Sequence
  Synthetic primer
taggggcagagggaatttcc

SEQ ID NO: 29 - DNA, 20 bp, Artificial Sequence
  Synthetic primer
ggtattattttagtggccca

SEQ ID NO: 30 - DNA, 20 bp, Artificial Sequence
  Synthetic primer
gcctgtgataataggaaagc

SEQ ID NO: 31 - DNA, 20 bp, Artificial Sequence
  Synthetic primer
agacggttttcagtgctagt

SEQ ID NO: 32 - DNA, 20 bp, Artificial Sequence
  Synthetic primer
tttggggaggggttttttac

SEQ ID NO: 33 - DNA, 35 bp, Artificial Sequence
  Synthetic primer
ggtgatatcccgcggatggtgagcaagggcgagga

SEQ ID NO: 34 - DNA, 35 bp, Artificial Sequence
  Synthetic primer
acgtaacatgccgggcttgtacagctcgtccatgc

SEQ ID NO: 35 - protein, 138 aa, Burkholderia cenocepacia
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRGATGETKVFTGNSNSPKSPTKGGC

SEQ ID NO: 36 - protein, 83 aa, Bacteriophage PBS2
TNLSDIIEKETGKQLVIQESILMLPEEVEEVIGNKPESDILVHTAYDESTDENVMLLTSDAPEYKPWALVIQDSNGENKIKML

SEQ ID NO: 37 - protein, 4 aa, Artificial Sequence
  Synthetic peptide
SGGS

SEQ ID NO: 38 - protein, 54 aa, Arabidopsis thaliana
MDSQLVLSLKLNPSFTPLSPLFPFTPCSSFSPSLRFSSCYSRRLYSPVTVYAAK

SEQ ID NO: 39 - DNA, 69 bp, Arabidopsis thaliana
cggggacttaacccaacacttacggcacgagctgacgacagccatgcaccacctgtgtccgcgttcccg

SEQ ID NO: 40 - DNA, 69 bp, Arabidopsis thaliana
gctcctgaattgggttgtgaatgccgtgctcgactgctgtcggtacgtggtggacacaggcgcaagggc

SEQ ID NO: 41 - DNA, 14 bp, Arabidopsis thaliana
agctgacgacagcc

SEQ ID NO: 42 - DNA, 14 bp, Arabidopsis thaliana
tcgactgctgtcgg

SEQ ID NO: 43 - DNA, 15 bp, Arabidopsis thaliana
tgaatcatataagaa

SEQ ID NO: 44 - DNA, 15 bp, Arabidopsis thaliana
agctagtatattctt

SEQ ID NO: 45 - DNA, 16 bp, Arabidopsis thaliana
aattgcagtcatggta

SEQ ID NO: 46 - DNA, 16 bp, Arabidopsis thaliana
ttaacgtcagtaccat

SEQ ID NO: 47 - DNA, 14 bp, Arabidopsis thaliana
agctgacgacagcc

SEQ ID NO: 48 - DNA, 14 bp, Arabidopsis thaliana
agctaacgacagcc

SEQ ID NO: 49 - DNA, 14 bp, Arabidopsis thaliana
agctaacgatagcc

SEQ ID NO: 50 - DNA, 14 bp, Arabidopsis thaliana
agctgacgacagcc

SEQ ID NO: 51 - DNA, 15 bp, Arabidopsis thaliana
tcaatcatataagaa

SEQ ID NO: 52 - DNA, 15 bp, Arabidopsis thaliana
tcgatcatataagaa

SEQ ID NO: 53 - DNA, 16 bp, Arabidopsis thaliana
aattgcagttatggta

SEQ ID NO: 54 - DNA, 16 bp, Arabidopsis thaliana
aattgcagtcatggta

SEQ ID NO: 55 - DNA, 14 bp, Arabidopsis thaliana
agttgacgatagcc

SEQ ID NO: 56 - DNA, 14 bp, Arabidopsis thaliana
agctaacgatagcc

SEQ ID NO: 57 - DNA, 14 bp, Arabidopsis thaliana
agctgacgacagcc

SEQ ID NO: 58 - DNA, 74 bp, Arabidopsis thaliana
cgctcagttgaaagctatgaaacaagtatgcggtagttcaaaactggaattggcacaatatcgcgaagtggccg

SEQ ID NO: 59 - DNA, 74 bp, Arabidopsis thaliana
gcgagtcaactttcgatactttgttcatacgccatcaagttttgaccttaaccgtgttatagcgcttcaccggc

SEQ ID NO: 60 - DNA, 16 bp, Arabidopsis thaliana
gcggtagttcaaaact

SEQ ID NO: 61 - DNA, 16 bp, Arabidopsis thaliana
cgccatcaagttttga

SEQ ID NO: 62 - DNA, 16 bp, Arabidopsis thaliana
gcggtagttcaaaact

SEQ ID NO: 63 - DNA, 16 bp, Arabidopsis thaliana
gcggtagtttaaaact

SEQ ID NO: 64 - DNA, 16 bp, Arabidopsis thaliana
gcgataatttaaaact

SEQ ID NO: 65 - DNA, 16 bp, Arabidopsis thaliana
gcggtagttcaaaact

SEQ ID NO: 66 - DNA, 16 bp, Arabidopsis thaliana
gcggtagttcaaaact

SEQ ID NO: 67 - DNA, 16 bp, Arabidopsis thaliana
gcggtagtttaaaact

SEQ ID NO: 68 - RNA, 22 nt, Arabidopsis thaliana
gcuaugaaacaaguaugcggua

SEQ ID NO: 69 - RNA, 71 nt, Arabidopsis thaliana
gucgggucugccgcucaguugaaagcuaugaaacaaguaugcgguaguucaaaacuggaauuggcacaaua

SEQ ID NO: 70 - DNA, 71 bp, Arabidopsis thaliana
gtcgggtctgccgctcagttgaaagctatgaaacaagtatgcggtagttcaaaactggaattggcacaata

SEQ ID NO: 71 - DNA, 71 bp, Arabidopsis thaliana; identical to SEQ ID NO: 70

SEQ ID NO: 72 - DNA, 71 bp, Arabidopsis thaliana
gtcgggtctgccgctcagttgaaagctataaaacaagtatgcggtagttcaaaactggaattggcacaata

SEQ ID NO: 73 - DNA, 71 bp, Arabidopsis thaliana
gtcgggtctgccgctcagttgaaagttataaaacaagtatgcggtagttcaaaactggaattggcacaata

SEQ ID NO: 74 - DNA, 71 bp, Arabidopsis thaliana
gtcgggtctgccgctcagttgaaagttatgaaacaagtatgcggtagttcaaaactggaattggcacaata

SEQ ID NO: 75 - DNA, 71 bp, Arabidopsis thaliana; identical to SEQ ID NO: 70

SEQ ID NO: 76 - DNA, 71 bp, Arabidopsis thaliana; identical to SEQ ID NO: 70

SEQ ID NO: 77 - DNA, 71 bp, Arabidopsis thaliana
gtcgggtctgccgctcagttgaaagctatgaaacaaatatgcggtagttcaaaactggaattggcacaata

SEQ ID NO: 78 - DNA, 71 bp, Arabidopsis thaliana; identical to SEQ ID NO: 70

SEQ ID NO: 79 - DNA, 71 bp, Arabidopsis thaliana; identical to SEQ ID NO: 70

SEQ ID NO: 80 - DNA, 71 bp, Arabidopsis thaliana
gtcgggtctgccgctcagttgaaagctatgaaacaagtatgcgatagttcaaaactggaattggcacaata

SEQ ID NO: 81 - DNA, 71 bp, Arabidopsis thaliana
gtcgggtctgccgctcagttgaaagctatgaaacaagtatgcaatagttcaaaactggaattggcacaata

SEQ ID NO: 82 - DNA, 16 bp, Arabidopsis thaliana
gcgatagtttaaaact

SEQ ID NO: 83 - DNA, 16 bp, Arabidopsis thaliana
gcggtagtttaaaact

SEQ ID NO: 84 - DNA, 16 bp, Arabidopsis thaliana
gcggtaatttaaaact

SEQ ID NO: 85 - DNA, 16 bp, Arabidopsis thaliana
gcggtagttcaaaact

SEQ ID NO: 86 - DNA, 16 bp, Arabidopsis thaliana
gcgatagtttaaaact

SEQ ID NO: 87 - DNA, 16 bp, Arabidopsis thaliana
gcggtagtttaaaact

SEQ ID NO: 88 - DNA, 16 bp, Arabidopsis thaliana
gcggtaatttaaaact

SEQ ID NO: 89 - DNA, 16 bp, Arabidopsis thaliana
gcggtagttcaaaact

SEQ ID NO: 90 - DNA, 16 bp, Arabidopsis thaliana
gcggtaattcaaaact

SEQ ID NO: 91 - DNA, 16 bp, Arabidopsis thaliana
gcggtagtttaaaact

SEQ ID NO: 92 - DNA, 16 bp, Arabidopsis thaliana
gcggtagtttaaaact

SEQ ID NO: 93 - DNA, 16 bp, Arabidopsis thaliana
gcgatagtttaaaact

SEQ ID NO: 94 - DNA, 16 bp, Arabidopsis thaliana
gcggtagttcaaaact

SEQ ID NO: 95 - DNA, 30 bp, Arabidopsis thaliana
gctatgaaacaagtatgcggttagttcaaa

SEQ ID NO: 96 - DNA, 30 bp, Arabidopsis thaliana
gctatgaaacaagtatgcggttagtttaaa

SEQ ID NO: 97 - DNA, 30 bp, Arabidopsis thaliana; identical to SEQ ID NO: 95

SEQ ID NO: 98 - DNA, 30 bp, Arabidopsis thaliana; identical to SEQ ID NO: 95

SEQ ID NO: 99 - DNA, 30 bp, Arabidopsis thaliana
gctataaaacaagtatgcggttagttcaaa

SEQ ID NO: 100 - DNA, 30 bp, Arabidopsis thaliana
gctataaaacaagtatgcggttagtttaaa

SEQ ID NO: 101 - DNA, 30 bp, Arabidopsis thaliana
gttataaaacaagtatgcggttagttcaaa

SEQ ID NO: 102 - DNA, 30 bp, Arabidopsis thaliana
gttataaaacaagtatgcggttagtttaaa

SEQ ID NO: 103 - DNA, 30 bp, Arabidopsis thaliana
gttatgaaacaagtatgcggttagttcaaa

SEQ ID NO: 104 - DNA, 30 bp, Arabidopsis thaliana
gttatgaaacaagtatgcggttagtttaaa

SEQ ID NO: 105 - DNA, 30 bp, Arabidopsis thaliana
gctatgaaacaagtatgcaattagttcaaa

SEQ ID NO: 106 - DNA, 30 bp, Arabidopsis thaliana; identical to SEQ ID NO: 105

SEQ ID NO: 107 - DNA, 30 bp, Arabidopsis thaliana; identical to SEQ ID NO: 105

SEQ ID NO: 108 - DNA, 30 bp, Arabidopsis thaliana; identical to SEQ ID NO: 105

SEQ ID NO: 109 - DNA, 30 bp, Arabidopsis thaliana; identical to SEQ ID NO: 105

SEQ ID NO: 110 - DNA, 30 bp, Arabidopsis thaliana; identical to SEQ ID NO: 105

SEQ ID NO: 111 - protein, 7 aa, Simian virus 40
PKKKRKV

SEQ ID NO: 112 - protein, 20 aa, Unknown
  nuclear localization signal/sequence
AVKRPAATKKAGQAKKKKLD

SEQ ID NO: 113 - protein, 25 aa, Unknown
  nuclear localization signal/sequence
MSRRRKANPTKLSENAKKLAKEVEN

SEQ ID NO: 114 - protein, 9 aa, Unknown
  nuclear localization signal/sequence
PAAKRVKLD

SEQ ID NO: 115 - protein, 9 aa, Unknown
  nuclear localization signal/sequence
KLKIKRPVK

SEQ ID NO: 116 - protein, 36 aa, Arabidopsis thaliana
MFKQASRLLSRSVAAASSKSVTTRAFSTELPSTLDS

SEQ ID NO: 117 - protein, 95 aa, Oryza sativa
MAARRAASSLLSRGLIARPSAASSTGDSAILGAGSARGFLPGSLHRFSAAPAAAATAAATEEPIQPPVDVKYTKLLINGNFVDAASGKTFATVDP

SEQ ID NO: 118 - protein, 54 aa, Pisum sativum
MWRRLFTSPHLKTLSSSSLSRPRSAVAGIRCVDLSRHVATQSAASVKKRVEDVV

(19) United States
(12) Patent Application Publication
(10) Pub. No.: US 2024/0218384 A1
(43) Pub. Date: Jul. 4, 2024

(54) METHOD FOR EDITING PLANT GENOME

(71) Applicant: The University of Tokyo, Tokyo (JP)

(72) Inventors: Shin-ichi ARIMURA, Tokyo (JP); Issei NAKAZATO, Kanagawa (JP); Nobuhiro TSUTSUMI, Chiba (JP); Ayako HOSODA, Tokyo (JP)

(73) Assignee: The University of Tokyo, Tokyo (JP)

(21) Appl. No.: 18/272,978

(22) PCT Filed: Jan. 21, 2022
(86) PCT No.: PCT/JP2022/002162

§ 371 Date: Jul. 18, 2023

Related U.S. Application Data
(60) Provisional application No. 63/285,223, filed on Dec. 2, 2021.

(30) Foreign Application Priority Data
Jan. 22, 2021 (JP) 2021-009001

(51) Int. Cl. C12N 15/82; C12N 9/78; C12N 15/62

(52) U.S. Cl. CPC C12N 15/8213; C12N 9/78; C12N 15/62; C07K 2319/02; C07K 2319/07; C07K 2319/09; C12Y 305/04005

(57) ABSTRACT

It is an object of the present invention to provide a method for editing or modifying plant genomes (a nuclear genome, a plastid genome, and a mitochondrial genome), and in particular, the editing or modification of a single nucleotide. Specifically, the present invention relates to a method for editing genomic DNAs in plant cells, namely, a nuclear genomic DNA, a plastid genomic DNA and a mitochondrial genomic DNA, wherein the method comprises converting target nucleotides on these genomic DNAs to other nucleotides. This conversion is carried out, for example, with cytidine deaminase, and in particular, with the aforementioned enzyme using a double-stranded DNA as a substrate.

### TECHNICAL FIELD

The present invention relates to a method for editing or modifying plant genomes, specifically, a nuclear genome, a mitochondrial genome, and a plastid genome.

### BACKGROUND ART

Upon selective breeding of higher plants, editing or modification of a nuclear genome is considered to be an effective method. In addition, genomes existing in plastids, including chloroplasts, and mitochondria, contain genes that play important roles, and editing of genomes contained in these intracellular organs, etc. is also considered to be effective for selective breeding of plants.

The plastid genome of higher plants has a size of about 150 kb and contains about 120 genes. These genes are associated with photosynthesis, antibiotic tolerance, herbicide tolerance, and the like. Among the plastid genes, for example, psbA, a key gene for photosystem, and rbcL, a key enzyme for dark-reaction CO₂ fixation, are important genes that carryout plant functions. It is expected that the improvement of these genes will contribute to optimization of light energy utilization in plants, the enhancement of food production, bioethanol production and increased biomass production, the improvement of CO₂ absorption and utilization as a resource, and the like.

Gene transfer into the plastid genome has been performed for about 30 years. The advantages of gene transfer into the plastid genome are different from those of gene transfer into the nuclear genome. For example, since the plastid genome is maternally inherited, it can prevent the spread of recombinant genes through pollens. In addition, the expression of a desired gene product is relatively easy because gene silencing, which occurs during the genetic recombination of the nucleus, does not occur.

However, the transfer of foreign genes into the plastid genome is not so easy. Special equipment (e.g., particle gun) and culture techniques are required for the gene transfer into the plastid genome. Moreover, the number of plant species, into which gene transfer can be carried out, is limited, and even in the case of model plants such as Arabidopsis thaliana and rice, it is difficult to transfer foreign genes into the chloroplast genome thereof (Non Patent Literature 1 and Non Patent Literature 2). Although there are some successful examples (for example, Patent Literature 1, etc.), gene transfer into the plastid genome is still a difficult technique.

Furthermore, to date, there are no practical techniques for genome editing that modifies only a specific single nucleotide in the plastid genome. The use of transgenic plants produced by the aforementioned gene transfer is internationally regulated by the Cartagena Act. In contrast, in some cases, the Cartagena Act may not apply to the modification of only a single nucleotide in the plastid genome that is originally present in plants, although the treatment is different from country to country. Therefore, it has been desired to develop a technique of modifying only a specific single nucleotide in the plastid genome, instead of gene transfer into the plastid genome.

The plant mitochondrial genome encodes not only genes involved in electron transport system, ATP synthesis, mitochondrial gene translation, etc., but also encodes many open reading frames (ORFs) whose functions are unknown. Insufficient utilization and characterization of the plant mitochondrial genome is partially caused by the limited tools for modification of the plant mitochondrial genome and the difficulty in identifying a single nucleotide polymorphism (SNP) in the genome that affects agronomic traits as a result of the modification. To date, stable gene transfer into the mitochondrial genome by a particle gun method has been performed on two unicellular organisms, namely, green alga Chlamydomonas (Non Patent Literature 3) and yeasts (Non Patent Literatures 4 and 5). However, stable gene transfer into the mitochondrial genome of higher plants has not been successfully achieved so far.

Recently, Mok et al. have bisected the cytidine deaminase (CD) gene of a Burkholderia cenocepacia DddA protein, and have fused an uracil glycosylase inhibitor (UGI) and the DNA-binding domain of TALE (transcription activator-like effector) with each of the obtained gene portions to create a protein, and thereafter, they have allowed the protein to transiently express in mammalian cells (Non Patent Literature 6). As a result, they have succeeded in substituting the target C:G pair in the mitochondrial genome with a T:A pair. The conversion of the C:G pair to the T:A pair has occurred in, at maximum, 50% of the mitochondrial genome in the cells.

Moreover, in order to replace the target base pair (conversion of C:G to T:A) in the mitochondrial genome of lettuce and rapeseed calluses, Kang et al. have applied the technique of Mok et al., and have allowed a fusion protein consisting of UGI and TALE to transiently express in the lettuce and rapeseed calli. As a result, Kang et al. have reported that the frequency of editing the mitochondrial genome is, at maximum, about 25% (Non Patent Literature 7).

As mentioned above, although the single nucleotide editing technique for plant genomes has been progressing year by year, its editing efficiency is still low at the present stage, and thus, further improvement of the technique is needed.

### CITATION LIST

### Patent Literature

Patent Literature 1: JP Patent Publication (Kokai) No. 2009-225721 A

### Non Patent Literature

Non Patent Literature 1: Yu et al., Plant physiology 175, 186-193, 2017.
Non Patent Literature 2: Ruf et al., Nature plants 5, 282-289, 2019.
Non Patent Literature 3: Remacle et al., Proc. Natl. Acad. Sci. 103, 4771-4776, 2006.
Non Patent Literature 4: Fox et al., Proc. Natl. Acad. Sci. 85, 7288-7292, 1988.
Non Patent Literature 5: Johnston et al., Science 240, 1538-1541, 1988.
Non Patent Literature 6: Mok et al., Nature 583, 631-637, 2020.
Non Patent Literature 7: Kang et al., Nat. Plants 7, 899-905, 2021.
Non Patent Literature 8: Gualberto et al., Biochimie 100, 107-120, 2014.
Non Patent Literature 9: Smith et al., Proc Natl Acad Sci USA 100, 892-897, 2003

### SUMMARY OF INVENTION

### Technical Problem

Under the aforementioned circumstances, it is an object of the present invention to provide a method for editing or modifying plant genomes, namely, a nuclear genome, a plastid (e.g., chloroplast) genome, and a mitochondrial genome in plants, and in particular, a method for editing or modifying a target single nucleotide with good accuracy and high efficiency.

### Solution to Problem

The present inventors have conducted intensive studies regarding whether the technique reported by Mok et al. (Non Patent Literature 6) could not be utilized for the editing of the nuclear genome, plastid genome, and mitochondrial genome of plants.

First, the present inventors have designed DNA-binding sequence TALE repeats used in the genome-editing enzyme TALEN (transcription activator-like effector nuclease), which recognizes 7 bp to 21 bp each before and after 10-20 bp containing a single nucleotide as a target of editing, and have then designed protein sequences (TALECD) by fusing the DNA-binding sequence TALE repeats with a half-split DddA Cytidine deaminase in each of the left and right pairs.

Subsequently, a nuclear transition (localization) signal (NLS) was added to these two proteins (nTALECD), a chloroplast transition (localization) signal was added to these two proteins (ptpTALECD), or a mitochondrial localization signal was added to these two proteins (mtpTALECD). Expression vectors for each protein (vectors that stably introduce DNA encoding each of the three types of peptide-added proteins into the nuclear genome) were constructed. These vectors were transformed into the nuclei of plant stem cells (DNA encoding each TALECD was incorporated into the plant nuclear genomic DNA, so that each of the above TALECDs can be expressed stably (not transiently). It could be confirmed that the nTALECD, ptpTALECD, or mtpTALECD expressed from these three types of expression vectors migrates into the nucleus, chloroplast, or mitochondria, respectively, and edits the target single nucleotide (conversion of C:G pair to T:A pair).

The present inventors have found that, by using the above-described method for editing a plant genome according to the present invention, the target C:G pairs contained in the plant genome (nuclear genome, plastid genome, and mitochondrial genome) can be homoplasmically modified, namely, if taking the plastid genome as an example, almost all of the target C:G pairs in about 1000 copies or more of plastid genomes contained in a cell in the plant can be converted to T:A pairs.

By the way, both plastids and mitochondria are cell organelles that are generated as a result of intracellular symbiosis of free-living bacteria, and retain their own genomic DNA. However, when compared with mitochondria, which have been intracellularly symbiotic for a longer period of time, the plastid genome has a sequence and a structure that are more similar to those of bacteria. In addition, unlike the mitochondrial genome, the plastid genome has transcription, translation, and DNA replication/repair systems that clearly exhibit bacterial types. Moreover, plant mitochondria duplicate and partially divert some of the enzymes of the DNA replication and repair system used in the plastid, and have their own hybrid-type system that is different from the plastid genome and the mammalian mitochondrial genome, which means that the three types of organellar genomes have three different styles. In fact, among the molecules identified as repair factors for plastid genomic DNA and mammalian mitochondrial genomic DNA, there are many completely different repair molecules. Therefore, genomic DNA repairs and changes that appear after modification of individual mitochondrial and plastid genomic DNAs are also different (see Non Patent Literature 8, Non Patent Literature 9, etc.).

As described above, since the mitochondria in mammals and the plastids and mitochondria in plants are completely different intracellular organelles, editing techniques applicable to the mitochondrial genome in mammals are not necessarily applicable to the editing of the mitochondrial genome and the plastid genome in plants.

Accordingly, the aforementioned results “the target C:G pairs can be homoplasmically modified” can be said to be significant effects that can never be predicted from the results disclosed in Non Patent Literature 6 that are “at most only about 42% of the target C:G pairs in mammalian cells was modified.” In addition, also regarding the technique of editing a mitochondrial genome and a plastid genome in plants disclosed in Non Patent Literature 7, the single nucleotide modification percentages were about 25% and about 38%, respectively. Taking into consideration these results, it can be said that the method for editing a plant genome according to the present invention is extremely efficient, compared with the method disclosed in Non Patent Literature 7.

Specifically, the present invention includes the following (1) to (6).
(1) A method for editing a plant genomic DNA, comprising converting a target nucleotide on the genomic DNA to another nucleotide. The conversion may be carried out with cytidine deaminase.
(2) In the above-described method for editing a plant genomic DNA, the cytidine deaminase may be a protein described in the following (a) or (b):
(a) a protein consisting of the amino acid sequence as set forth in SEQ ID NO: 35; or
(b) a protein consisting of an amino acid sequence having a sequence identity of 90% or more to the amino acid sequence as set forth in SEQ ID NO: 35, and having cytidine deaminase activity.
(3) In the above-described method for editing a plant genomic DNA, an N-terminal portion of the cytidine deaminase and the other portion may be each fused with a different TALE (transcription activator-like effector).
(4) The above-described method for editing a plant genomic DNA may be a method comprising introducing a DNA encoding a fusion protein consisting of a part of or the entire cytidine deaminase and TALE, to which a nuclear localization signal peptide, a plastid localization signal peptide or a mitochondrial localization signal peptide is added (i.e. a DNA encoding the fusion protein), into a nuclear genome in a plant cell (i.e. incorporating the DNA into the nuclear genomic DNA), and then allowing the signal peptide-added fusion protein to express in the plant cell, so that a target nucleotide in a nuclear genomic DNA, a plastid genomic DNA or a mitochondrial genomic DNA in a plant is converted to another nucleotide.
(5) A plant genome comprising a plant genomic DNA edited by the above-described method for editing a plant genomic DNA, a plant cell having the plant genome, and a seed or a plant comprising the plant cell.
(6) A method for producing a plant having an edited plant genome, wherein the method comprises editing the plant genome by the method for editing a plant genomic DNA according to any one of the above (1) to (4).

It is to be noted that the preposition “to” sandwiched between numerical values is used in the present description to mean a numerical value range including the numerical values located left and right of the preposition.

### Advantageous Effects of Invention

According to the method of the present invention, it is possible to modify a single nucleotide in a plant genome, specifically, in a nuclear genome, a plastid genome or a mitochondrial genome in a plant. Moreover, according to the method of the present invention, target nucleotides of almost all of copies of a nuclear genome, a plastid genome or a mitochondrial genome in a plant body can be modified.

### BRIEF DESCRIPTION OF DRAWINGS

FIGS. 1a and 1b show the action mechanism of ptpTALECD, which targets a plastid gene, and an expression vector therefor. FIG. 1a schematically shows the target region in pTALECD and 16S rRNA genes. The 16S rRNA sequences shown in the figure are SEQ ID NO: 39 and SEQ ID NO: 40 from above. FIG. 1b shows the T-DNA region of the tandem expression vector of ptpTALECD. “1333C” is a protein consisting of the amino acid sequence from the 45th to the 138th amino acids on the C-terminal side of the amino acid sequence of DddAₜₒₓ as set forth in SEQ ID NO: 35, and “1333N” is a protein consisting of the amino acid sequence from the 1st to the 44th amino acids on the N-terminal side of the amino acid sequence of DddAₜₒₓ as set forth in SEQ ID NO: 35. “1397C” is a protein consisting of the amino acid sequence from the 95th to the 138th amino acids on the C-terminal side of the amino acid sequence of DddAₜₒₓ as set forth in SEQ ID NO: 35, and “1397N” is a protein consisting of the amino acid sequence from the 1st to the 94th amino acids on the N-terminal side of the amino acid sequence of DddAₜₒₓ as set forth in SEQ ID NO: 35.

FIGS. 2a and 2b are schematic views showing a step of constructing a ptpTALECD expression vector. FIG. 2a shows assembly steps for constructing a pTALECD ORF. Basically, Platinum TALEN Kit was used, but an entry vector in step 2 was produced by the process shown in FIG. 8. FIG. 2b shows the process of constructing a ptpTALECD expression vector. The ptpTALECD expression vector was constructed using LR Clonase™ II Plus enzyme (Thermo Fisher Scientific).

FIG. 3 shows the replacement of a FokI coding sequence with the coding sequence of one side (herein referred to as a “CD half”) obtained by dividing cytidine deaminase (i.e., DddAₜₒₓ). The FokI coding sequence and the coding sequence of the CD half (SEQ ID NOs: 7 to 10) inserted into the entry vector of step 2 used in Arimura et al. The Plant Journal 2020, 104, 1459-1471 were amplified by PCR. The purified PCR amplified products were mixed with 5× In-Fusion HD Cloning Enzyme Premix (TaKaRa) and were then incubated at 50° C. for 15 minutes.

FIGS. 4a to 4g show the result of editing cytidine in the target region. FIGS. 4a to 4c show the number of individual plants having cytidine nucleotide substitutions, the editing efficiency, and the predicted amino acid substitutions. The sequences shown in FIG. 4a are SEQ ID NO: 41 and SEQ ID NO: 42 from above: the sequences shown in FIG. 4b are SEQ ID NO: 43 and SEQ ID NO: 44 from above: and the sequences shown in FIG. 4c are SEQ ID NO: 45 and SEQ ID NO: 46 from above. FIGS. 4d-f show representative analysis results of Sanger sequencing of ptpTALECD target sequences in T₁ plants at 23 days after the stratification treatment of dormancy awakening (hereinafter referred to as “23 DAS”). The sequences shown in FIG. 4d are SEQ ID NO: 47, SEQ ID NO: 47, SEQ ID NO: 48, SEQ ID NO: 49, and SEQ ID NO: 50 from above: the sequences shown in FIG. 4e are SEQ ID NO: 51, SEQ ID NO: 52, SEQ ID NO: 51, and SEQ ID NO: 52 from above: and the sequences shown in FIG. 4f are SEQ ID NO: 53, SEQ ID NO: 53, and SEQ ID NO: 54 from above. FIG. 4g shows the number of plants, which is summarized by substitution mutation types of the target nucleotides of T₁ plants at 11 DAS and 23 DAS. h/c (heteroplasmically or chimerically): heteroplasmic or chimeric substitution: homo: homoplasmic substitution: Cp: target cytosine for which a preferential substitution is predicted: and Cp*: cytosine predicted to cause biological effects.

FIGS. 5a and 5b show the results of the analysis of chimerically nucleotide-edited leaves. FIG. 5a shows a leaf image of 16SrRNA 1397NC (1397N-1397C) line 3 at 23 DAS showing partially different color schemes. FIG. 5b shows the results of the analysis of the genotype of a ptpTALECD target region. The sequences shown in FIG. 5b are SEQ ID NO: 55, SEQ ID NO: 56, and SEQ ID NO: 57 from above.

FIGS. 6a to 6d show the results of the analysis of a T₂ generation, namely, the genotypes and phenotypes of six T₂ plants of 16S rRNA1397CN line 2. The upper view of FIG. 6a shows the results of PCR amplification of the GFP and the target sequence 16S rRNA of three plants each whose seeds were GFP positive and negative (i.e., three plants that inherited a T-DNA vector in the nucleus thereof (positive) and three plants whose seeds did not inherit the T-DNA vector (negative)): and the lower view of FIG. 6a shows the genotypic analysis results of G₅ single nucleotide polymorphisms (SNPs) and phenotypes thereof. FIG. 6b shows the representative phenotypes of the T₂ generation of 16S TRNA1397CN line 2. The bar indicates 1 mm. FIGS. 6c and d show the phenotypes of the T₂ generation of 16S rRNA1397CN line 2 and 16S rRNA1397CN line 15 in the presence of Spm (spectinomycin). FIG. 6c shows images of T₂ generation of the two lines on a ½ MS medium containing 50 mg/L Spm (spectinomycin) and wild type seeds (0 DAS) and seedlings (8 DAS). FIG. 6d shows the results obtained by summarizing the relationship between the presence or absence of GFP fluorescence in seeds and the color of 8 DAS plants. W/G: plants with white or red cotyledons and green true leaves; n.g.: not germinated.

FIGS. 7a and 7b show the results of the analysis of the genotypes and phenotypes of T₂ plants. FIG. 7a shows the results of summarization of the genotypes and phenotypes of T₂ plants, which were obtained by the inbreeding of 16S rRNA1397CN line 2, line 8, and 1397NC line 3. FIG. 7b shows the representative phenotypic images of the T₂ plants shown in FIG. 7a. The bar indicates 0.5 mm.

FIGS. 8a and 8b show construction of a 2^{nd} entry vector and a destination vector. FIG. 8a shows the process of constructing a 2^{nd} entry vector. The 2^{nd} entry vector (used in Arimura et al., The Plant Journal 104, 1459-1471, 2020) and a RECA1 plastid localization peptide coding sequence were amplified by PCR. The purified PCR amplified product was mixed with 5× In-Fusion HD Cloning Enzyme Premix (TaKaRa), and the obtained mixture was then incubated at 50° C. for 15 minutes. FIG. 8b shows the process of constructing a destination vector. The destination vector (used in Arimura et al., The Plant Journal 104, 1459-1471, 2020) was amplified by PCR. The purified PCR amplified product was mixed with 5× In-Fusion HD Cloning Enzyme Premix (TaKaRa), and the obtained mixture was then incubated at 50° C. for 15 minutes. The assembled destination vector was cleaved with KpnI, and the purified product was mixed with 5× In-Fusion HD Cloning Enzyme Premix (TaKaRa) and the OLE1GFP coding sequence amplified from pFAST02 (INPLANTAINNOVATIONS INC). The thus obtained mixture was incubated at 50° C. for 15 minutes to construct a ptpTALECD expression vector.

FIG. 9 shows the genotypes of the cotyledons of Spm^{r} (spectinomycin-resistant) plants and Spm^{s}-like (spectinomycin-sensitive-like) plants at 13 DAS. FIG. 9 shows the presence or absence of seed GFP fluorescence, the presence or absence of G₅ SNP, and the phenotypes of Spm^{r} plants (T₂ of 16S rRNA1397CN line 15) and Spm^{s}-like plants (T₂ of 16S rRNA1397CN line 2) shown in FIG. 6c at 13 DAS. W/G: White or red cotyledons and green true leaves.

FIGS. 10a to 10d show introduction of a homoplasmic mutation into the target nucleotide in apt1. FIG. 10a schematically shows a pair of pTALECD proteins, a target nucleotide, and a target region. For the divided position of CD, refer to the explanation of FIG. 1. The N-terminal half CD and the C-terminal half CD were each fused with TALE. UGI: uracil glycosylase inhibitor. The sequences shown in FIG. 10a are SEQ ID NO: 58 and SEQ ID NO: 59 from above. FIG. 10b shows the number of plants with cytidine nucleotide substitution in T₁ plants at 11 days after a stratification treatment of dormancy awakening (11 DAS), editing efficiency, and predicted amino acid substitution. Cp: C at the T position of the 3′ side chain: Cp*: special target of otp87: No.: the number of total T₁ plants: h/c: heteroplasmic and/or chimeric substitution: and homo: homoplasmic substitution. The sequences shown in FIG. 10b are SEQ ID NO: 60 and SEQ ID NO: 61 from above. FIG. 10c shows 4 representative examples of Sanger sequencing of the amplified PCR products of the target sequences. The sequences shown in FIG. 10c are SEQ ID NO: 62, SEQ ID NO: 63, SEQ ID NO: 64, and SEQ ID NO: 65 from above. FIG. 10d shows the number of plants, which is summarized by substitution mutation types of the target nucleotides of T₁ plants at 11 DAS and 23 DAS. The mutation stability percentage (%) is calculated by dividing the number of nucleotides with changed mutations by the total number of substituted nucleotides. An “unstable” mutation means that the type of mutation differs between plants at 11 DAS and those at 23 DAS.

FIGS. 11a to 11c show the results of the analysis of T₂ plants. FIG. 11a shows the genotypes of the T₂ generation of eight plants of atp1 1397NC 4. T-DNA-derived seed-specific GFP expression was confirmed by fluorescence. The positive signal of mtpTALECD amplification indicates that the mtpTALECD gene introduced into the nuclear genome was inherited. atp1 is a positive control to PCR amplification of mtpTALECD. Sanger data for two nucleotides in the target window (G4 and C10: positions where the parent plant has a mutation) are shown in the lower view. NTC: no template control (a control without addition of a template). FIG. 11b shows the genotypes of the T₂ generation of 4 lines at 20 DAS, Col-0, and otp87. Five nuclear mtpTALECD gene-free T₂ generations (T₂ no. 9-13 shown in FIG. 16 and FIG. 17) of 4 T₁ lines (atp1 1333CN 3, 1333NC 7, 1397CN 24, and 1397NC 4) inherited a mitochondrial homoplasmic mutation, and grew at the same level as Col-0 and grew better than otp87. The bar indicates 1 cm. FIG. 11c shows the results of the analysis of on-target and off-target SNPs in the mitochondrial genomes of 8 representative T₂ plants (2 descendants from each of 4 T₁ lines). None of these plants contained the mtpTALECD gene. The X-axis and the Y-axis show the position and frequency of mutated SNPs (≥5% different from the reference genome (BK010421.1)). The allele frequency was calculated by AFₘᵤ-AF_{WT}. AFₘᵤ is the allele frequency of SNPs for each mutation, and AF_{WT} is the mean value of the same SNPs in the three wild-type plants.

FIG. 12 shows the repair of mitochondrial atp1 RNA in an otp87 mutant by mtpTALECD. The left views show representative examples of individual plants at 13 DAS of Col-0, the otp87 mutant, and the otp87 with atp1 modified by mtpTALECD. The right views show the DNA and RNA sequences around 393Leu of atp1. In the uppermost view, C in the 393Leu codon is usually converted to T according to RNA editing by otp87. In the otp87 mutant (middle view), this conversion does not occur, and substitution of Leu to Ser occurs, which prevents the growth of individual plants. In order to restore the normal growth of the mutant, C in atp1 was substituted with T, using mtpTALECD (lowermost view). In this case, the RNA editing by OTP87 was not necessary. This substitution restored the growth of the otp87 mutant up to the same level as that of a wild type. Other experimental results are shown in FIGS. 21a and 21b. The bar indicates 1 cm. The sequences shown in the figures are SEQ ID NO: 66, SEQ ID NO: 67, SEQ ID NO: 66, SEQ ID NO: 66, SEQ ID NO: 67, and SEQ ID NO: 67 from above.

FIGS. 13a to 13c show the effects of mutations in the predicted OTP87 binding sequence in the atp1 sequence on the RNA editing by OTP87. FIG. 13a shows the RNA sequence logo showing the probability of occurrence of the nucleotides to which each PPR motif of OTP87 binds based on the two important amino acids at positions 5 and 35 of each PPR motif of OTP87. The actual RNA sequence corresponding to the predicted binding site is located upstream of the RNA editing site by OTP87 in atp1 (A shows the sequence (SEQ ID NO: 68)). The PPR motifs are numbered from the C-terminal amino acid. The C-terminal S2 domain and the N-terminal S domain correspond to the 4th nucleotide (-4A) and the nucleotide 25 upstream from the editing site (-25G), respectively. The target nucleotide of mtpTALECD (see the explanation of FIG. 13b) is circled with the square. FIG. 13b shows the RNA sequence of the predicted binding site of OTP87 in atp1 and the RNA editing site (see the uppermost sequence). In the sequence, -20G, -13G and -6G were substituted with A by three pairs of mtpTALECD, respectively. In addition, the alleles obtained by the editing, the plant number of each allele, and the RNA editing from 1178C to U are also shown. The TALE-binding sequences are underlined. h/c (heteroplasmically or chimerically): heteroplasmic or chimeric substitution: and homo: homoplasmic substitution. Besides, the sequences shown in FIG. 13b are SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, and SEQ ID NO: 81 from above. FIG. 13c shows representative examples of RNA (complementary DNA) sequences around the RNA editing sites of the obtained alleles. In FIG. 13c, the lowermost example shows the data of an example, in which C was converted to T(U) at the highest level among the five (little) edited plants (i.e. it shows that RNA editing hardly occurs among these plants). Images of all analyzed plants and the genotypes thereof are shown in FIGS. 22b and 22c, and FIG. 23.

FIG. 14 shows a schematic view of a mtpTALECD tandem expression vector. The primers used in FIG. 11a are shown.

FIG. 15 shows the results (1) of Sanger sequencing of amplicons amplified with primers that bind to both nuclear mitochondrial (NUMT) DNA sequences and mitochondrial DNA sequences. Representative examples of the Sanger sequencing results of PCR amplified products that were amplified using the primers that bind to both nuclear mitochondrial DNA sequences and mitochondrial DNA sequences (left side), and primers that bind specifically to mitochondrial DNA (right side), are shown. The data shown in the same position on the left and right are the results of an identical plant. h/c (heteroplasmically or chimerically): heteroplasmic or chimeric substitution: and homo: homoplasmic substitution. (That is, the figure shows that, in these plants, the mitochondrial DNA is homoplasmically edited, and at the same time, homologous sequences exist in the nucleus, but these sequences are not edited.) The sequences shown in the figure are SEQ ID NO: 82, SEQ ID NO: 83, SEQ ID NO: 84 and SEQ ID NO: 85 from above left, and are SEQ ID NO: 86, SEQ ID NO: 87, SEQ ID NO: 88 and SEQ ID NO: 89 from above right.

FIG. 16 shows the results (2) of Sanger sequencing of amplicons amplified with primers that bind to both nuclear mitochondrial (NUMT) DNA sequences and mitochondrial DNA sequences. A genotype list of 11 DAS and 23 DAS is shown. *: DNA was extracted from cotyledons. **: According to these nucleotide substitutions, the amino acid G is substituted with N (when the nucleotides G3 and G4 are substituted with A), with S (when only G3 is substituted with A), or with D (when only G4 is substituted with A). n.e.: not analyzed.

FIG. 17 shows the results (3) of Sanger sequencing of amplicons amplified with primers that bind to both nuclear mitochondrial (NUMT) DNA sequences and mitochondrial DNA sequences. A genotype list of 11 DAS and 23 DAS is shown. **: According to these nucleotide substitutions, the amino acid G is substituted with N (when the nucleotides G3 and G4 are substituted with A), with S (when only G3 is substituted with A), or with D (when only G4 is substituted with A).

FIG. 18 shows the genotypes of T₂ plants. The results of the DNA sequencing of the target regions of T₂ plants are shown. Primers specific to the mitochondrial genome (primers that do not amplify NUMT) were used for PCR. The rightmost column shows the results of the Sanger sequencing of the target regions of 13 representative plants (number 9) of each line. Several nucleotides that had been homoplasmically and/or heteroplasmically mutated in the T₁ generation were changed to uniform genotypes in the T₂ generation. For example, in 1397CN 24, G4 was h/c at 11 DAS in the T₁ generation, but in the T₂ generation, it was reverted to the wild type. The sequences shown in the rightmost column are SEQ ID NO: 90, SEQ ID NO: 91, SEQ ID NO: 92, and SEQ ID NO: 93 from above. * The genotypes of T₁ are identical at both 11 DAS and 23 DAS. ** The genotypes of plants (numbers 9 to 13 in each line) are genotypes at 20 DAS. h/c (heteroplasmically or chimerically): heteroplasmic or chimeric substitution: and homo: homoplasmic substitution.

FIG. 19 shows a comparison of mitochondrial genome coverage analysis patterns of NGS short reads, which were obtained from T₂ plants treated with mitoTALEN and mtpTALECD. A coverage cover of T₂ plants treated with mitoTALEN was obtained from a previous report (Arimura et al., Plant J. 104, 1459-1471, 2020). The sequence information is the same as that given in FIG. 2c. A narrow gap common in all of the plants including Col-0 is an artifact caused by the removal of reads homologous to the sequences in the plastid genome. The white and black circles shown in the figure indicate the target sites of mtpTALECD and mitoTALEN, respectively.

FIG. 20 shows the amplicon sequencing of the atp1-like NUMT sequences of T₂ plants. The plants with Nos. 9-12 from each of the 4 lines were selected as representative examples. The C corresponding to 1178C of atp1 is indicated with the arrow. The sequencing results demonstrate that no important substitutions occurred in sequences homologous to the target region. The sequences shown in the figure are all SEQ ID NO: 94.

FIGS. 21a and 21b show the growth status and genotypes of T₁ otp87 transformed with atp1 1397CN. FIG. 21a shows an image of individual plants at 13 DAS. The bar indicates 1 cm. FIG. 21b shows the genotypes of the T₁ plants shown in FIG. 21a.

FIGS. 22a to 22c show the phenotypes and genotypes (1) of all analyzed T₁ plants, among T₁ plants whose predicted OTP87-binding sequences were edited. FIG. 22a shows the predicted OTP87-binding RNA sequence in apt1 and the RNA editing site thereof. The amino acid sequence substitution induced by the conversion of C:G to T:A by mtpTALECD and the RNA editing are shown. FIG. 22b shows the appearances of all plants analyzed at 12 DAS. FIG. 22c shows the genotypes of the T₁ plants shown in FIG. 22b. Only the data regarding plants confirmed to have a mutation, out of the 15 plants, are shown.

FIG. 23 shows the phenotypes and genotypes (2) of all analyzed T₁ plants, among T₁ plants whose predicted OTP87-binding sequences were edited. Representative examples of the Sanger sequencing of mutant alleles and the presence or absence of 1178CRNA editing are shown. The sequences shown in the figure are SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, and SEQ ID NO: 110 from above.

FIG. 24 shows the editing of the CYO1 gene by nTALECD. FIG. 24a shows representative examples of the phenotypes of a cyo1 mutant and a wild type at the time of true leaf emergence (11 DAS). FIGS. 24b to d show representative examples of the phenotypes at 7 DAS of T₁ generation, into which nTALECD was introduced. FIG. 24e shows the phenotypes of the T₁ generation cotyledons (7 DAS), into which nTALECD was introduced. FIG. 24f shows the number of plants for the phenotypes of T₁ plant population and WT plant population of CYO1 ex1 (example 1) and ex2 (example 2). DAS: Days after stratification.

FIG. 25 shows site-specific nucleotide substitutions introduced into the target sequences in CYO1. The number of transgenic plants per nucleotide of the CYO1 ex1/ex2 target sequence examined by PCR Sanger sequencing at the time of 21 DAS is shown. h/c: Heterozygote or chimera of a wild type and a mutant type. In both ex1 and ex2, these mutations form stop codons (ex1: CGA to TGA; and ex2: TGG to TGA or TAG or TAA).

FIG. 26 shows site-specific nucleotide substitutions introduced into the target sequences in PKT3 or MSH1. The number of transgenic plants per nucleotide examined by PCR Sanger sequencing at the time of 21 DAS is shown. h/c: Heterozygote or chimera of a wild type and a mutant type.

FIG. 27 shows studies regarding the presence or absence of off-target editing around the target sequence. The off-target mutation information in the region around 200 bp (a) and 1 kbp (b) of the target sequence examined by PCR Sanger sequencing at the time of 35 DAS, and the ratio of the number of plants with a mutation detected to the number of examined plants, are shown.

### DESCRIPTION OF EMBODIMENTS

Hereafter, the embodiments for carrying out the present invention will be described.

A first embodiment relates to a method for editing a plant genomic DNA, comprising converting a target nucleotide on the genomic DNA to another nucleotide.

In the present embodiment, the “plant genome” means a genome contained in the nucleus of a plant (nuclear genome), a genome contained in the plastid of a plant (plastid genome), or a genome contained in the mitochondria of a plant (mitochondrial genome). In addition, in the present embodiment, the “plastid” means an organelle present in the cells of plants, algae and the like, and the plastid performs anabolism such as photosynthesis, the storage of sugars, fats, etc., and the synthesis of various compounds. Examples of the “plastid” may include chloroplasts, leucoplasts, and chromoplasts.

Modification of a target nucleotide is not particularly limited, but it may be carried out using a nucleotide-modifying enzyme such as deaminase that is introduced into the nucleus, plastid, or mitochondria. Such an enzyme may be, for example, cytidine deaminase that converts the cytosine (C) in DNA to uridine (U). The enzyme is particularly preferably an enzyme that converts the C in double-stranded DNA to U, and it is, for example, a cytidine deaminase domain of DddA of Burkholderia cenocepacia (hereinafter referred to as “DddAₜₒₓ”: SEQ ID NO: 35), or a protein substantially identical to DddAₜₒₓ. In this context, the protein substantially identical to DddAₜₒₓ is not particularly limited, and it is, for example, a protein comprising an amino acid sequence having an amino acid identity of 70% or more, preferably 80% or more, more preferably 90% or more, 91% or more, 92% or more, 93% or more, 94% or more, 95% or more, 96% or more, 97% or more, 98% or more, and most preferably 99% or more, to the amino acid sequence as set forth in SEQ ID NO: 35, and having cytidine deaminase activity (the activity of converting the C in double-stranded DNA to U).

In order to specifically modify the target nucleotide of a nuclear genomic DNA, plastid genomic DNA, or mitochondrial genomic DNA in plants, it is necessary to allow a modifying enzyme such as deaminase (for example, cytidine deaminase) to recognize the target nucleotide. As a means therefore, there may be applied a method comprising: ligating a modifying enzyme to TALE (transcription activator-like effector) that binds to a genomic DNA around the target nucleotide (for example, within a range of 0 to 1000 nucleotides, preferably 5 to 100 nucleotides, and more preferably 5 to 50 nucleotides, from the target nucleotide): and then introducing the modifying enzyme-TALE fusion protein into the nucleus, plastid or mitochondria in plants. More specifically, for example, a DNA encoding such a modifying enzyme-TALE fusion protein may be introduced into a nuclear genomic DNA (may be incorporated into the nuclear genomic DNA), and thereafter, the modifying enzyme-TALE fusion protein expressed in the cytoplasm may be transported (introduced) into the nucleus, plastid, or mitochondria. In this case, it is desirable to introduce a DNA encoding a fusion protein formed by adding (binding) a different type of signal peptide (a nuclear localization signal peptide, a plastid localization signal peptide, or a mitochondrial localization signal peptide) as described below to the modifying enzyme-TALE fusion protein, into the nuclear genomic DNA.

As a method of transporting the modifying enzyme-TALE fusion protein into the nucleus, there can be applied a method which comprises fusing the modifying enzyme-TALE fusion protein with a nuclear localization signal/sequence (NLS) peptide, and then expressing the fused body. Examples of the nuclear localization signal peptide usable in the embodiment of the present invention may include, but are not limited to, an SV40 large T antigen NLS peptide (PKKKRKV, SEQ ID NO: 111), a nucleoplasmin NLS peptide (AVKRPAATKKAGQAKKKKLD, SEQ ID NO: 112), an EGL-13 NLS peptide (MSRRRKANPTKLSENAKKLAKEVEN, SEQ ID NO: 113), a c-Myc NLS peptide (PAAKRVKLD, SEQ ID NO: 114), and a TUS protein NLS peptide (KLKIKRPVK, SEQ ID NO: 115). Other than these NLS peptides, usable nuclear localization signal peptides are present, and see, for example, NLSdb (https://rostlab.org/services/nlsdb/browse/signals) that is the database of nuclear localization signals.

As a method of transporting the modifying enzyme-TALE fusion protein into the plastid, there can be applied a method which comprises fusing the modifying enzyme-TALE fusion protein with a plastid localization signal peptide (a peptide that has neither a clear higher-order structure nor sequence homology, but is rich in basic amino acids and multiple hydrophobic amino acids, contains a few acidic amino acids, and exhibits the function of specifically sorting and transporting to chloroplasts or plastids by adding it to the N-terminus of the amino acid sequence of the protein), and then expressing the fused body. The plastid localization signal peptide usable in the embodiment of the present invention is preferably a signal peptide possessed by a protein localized in a plant plastid. Examples of a preferred signal peptide may include, but are not limited to, protein-derived signal peptides such as RECA1, RBCS, CAB, NEP, SIG1 to 5, and GUN2 to 5, nuclear-encoded chloroplast ribosomal protein-derived signal peptides such as RPL12 and RPS9, nuclear-encoded chloroplast tRNA aminoacyl transferase-derived signal peptides, nuclear-encoded chloroplast heat shock protein-derived signal peptides, protein-derived signal peptides such as FtsZ, FtsH, MinC, MinD, and MinE, nuclear-encoded chloroplast photosynthesis-related enzyme complex group-derived signal peptides, nuclear-encoded plastid lipid metabolism enzyme group-derived signal peptides, and nuclear-encoded thylakoid protein group-derived signal peptides. For the plastid localization signal peptides, see, for example, von HEIJNE et al., Eur. J. Biochem. 180, 535-545, 1989.

As a method of transporting the modifying enzyme-TALE fusion protein into the mitochondria, there can be applied a method which comprises fusing the modifying enzyme-TALE fusion protein with a mitochondrial localization signal peptide (a peptide that does not have a clear higher-order structure or sequence homology, but is characterized in that, for example, basic amino acids and multiple hydrophobic amino acids appear alternately), and then expressing the fused body. The plastid localization signal peptide usable in the embodiment of the present invention may preferably be, for example, a signal peptide possessed by a protein localized in plant mitochondria. Examples of the preferred signal peptide may include, but are not limited to, an Arabidopsis thaliana ATPase δ′ subunit-derived signal peptide (MFKQASRLLS RSVAAASSKS VTTRAFSTEL PSTLDS, SEQ ID NO: 116), a rice ALDH2a gene product-derived signal peptide (MAARRAASSL LSRGLIARPS AASSTGDSAI LGAGSARGFL PGSLHRFSAA PAAAATAAAT EEPIQPPVDV KYTKLLINGN FVDAASGKTF ATVDP, SEQ ID NO: 117), a pea cytochrome c oxidase Vb-3-derived signal peptide (MWRRLFTSPH LKTLSSSSLS RPRSAVAGIR CVDLSRHVAT QSAASVKKRV EDVV, SEQ ID NO: 118), an Arabidopsis thaliana ATPase β subunit-derived signal peptide, a chaperonin CPN-60-derived signal peptide (Logan et al., Journal of Experimental Botany 50, 865-871, 2000), a rice ALDH signal peptide (Nakazono et al., Plant Physiology 124, 587-598, 2000), and a rice FIFO-ATPase inhibitor protein signal peptide (Nakazono et al., Plant 210, 188-194, 2000).

Otherwise, it is also possible to use a method which comprises directly introducing a plasmid DNA or mRNA encoding the modifying enzyme-TALE fusion protein, and the modifying enzyme-TALE fusion protein, and the like into a cell (wherein examples of the introduction method may include a virus method, a particle gun method, a PEG method, and a cell membrane-penetrating peptide method).

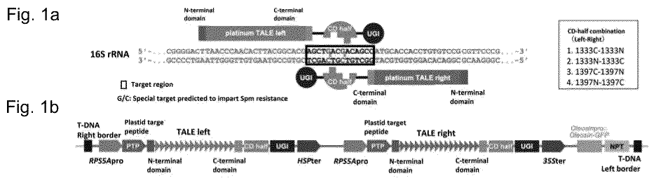

In order to modify a target nucleotide in a plant genomic DNA with high probability, two modifying enzyme-TALE fusion proteins (for example, the TALE left and TALE right shown in FIG. 1, in which modification of a plastid genome is taken as an example) may be simultaneously expressed in a single Ti plasmid, and also, for localization in a nucleus, a plastid or mitochondria, a tandem expression Ti plasmid, to which a nuclear localization signal peptide, a plastid localization signal peptide or a mitochondrial localization signal peptide is added, may be used (see, for example, Non Patent Literature 6).

Moreover, when a full-length protein such as DddAₜₒₓ is used as an enzyme for modifying the target sequence, if the direct use thereof affects the cells due to its toxicity, partial proteins prepared by dividing such a full-length protein at an appropriate position may be each fused with the aforementioned TALE left and TALE right, and each fusion protein may be then transferred into the plastid. The two partial proteins, which are obtained by dividing the full-length protein at the appropriate position, can be reassociated with each other at a stage in which they bind to the vicinity of the target nucleotide, and can exhibit desired activity (see the Examples). When DddAₜₒₓ is used as a modifying enzyme, for example, the amino acid sequence of DddAₜₒₓ as set forth in SEQ ID NO: 35 may be divided between any amino acids at positions 40 to 100, for example, between the amino acids at positions 44 and 45, or between the amino acids at positions 94 and 95.

Furthermore, the modifying enzyme-TALE fusion protein may be fused with other proteins that have functions to enhance the action of the fusion protein. An example of such other proteins may be an uracil glycosylase inhibitor (UGI). UGI inhibits the activity of uracil glycosylase, which removes U. Accordingly, when cytidine deaminase is used as a modifying enzyme, UGI plays a role of preventing the removal of U that is converted from C, and maintaining the modification by the cytidine deaminase-TALE fusion protein.

In the first embodiment, for example, if the aforementioned cytidine deaminase (CD), DddAₜₒₓ, is used as a modifying enzyme, the target nucleotide C in a nuclear genomic DNA, a plastid genomic DNA and a mitochondrial genomic DNA can be converted to T, homoplasmically (a state in which the same mutations are kept in all of cells and tissues, or in plants). Therefore, the present invention provides an extremely useful means for improving plants.

A second embodiment relates to: a nuclear genome in which a target nucleotide in the nuclear genomic DNA of a plant is modified, a plastid genome in which a target nucleotide in the plastid genomic DNA of a plant is modified, or a mitochondrial genome in which a target nucleotide in the mitochondrial genome DNA of a plant is modified, wherein the modification is carried out by the method for editing a plant genomic DNA according to the first embodiment; a nucleus having the nuclear genome, a plastid having the plastid genome, or mitochondria having the mitochondrial genome: a plant cell having the nuclear genome, the plastid genome or the mitochondrial genome: a cytoplasm of the plant cell: or a seed or a plant (an adult plant), comprising the plant cell.

The plant (adult plant) in the present embodiment includes not only generations (T₀, or also, T₁ depending on the plant type) that are differentiated from transformed cells, in which a target nucleotide in a nuclear genomic DNA, a target nucleotide in a plastid genomic DNA, or a target nucleotide in a mitochondrial genomic DNA is modified, but also includes generations of progenies obtained from T₀/T₁. In addition, the seeds in the second embodiment include not only seeds obtained from the above-described T₀/T₁ generations, but also include seeds obtained from the generations of progenies.

A third embodiment relates to a method for producing a plant having an edited plant genome, wherein the method comprises editing a plant genome by the method for editing a plant genomic DNA according to the first embodiment.

That is to say, the third embodiment relates to:

a method for producing a plant having an edited nuclear genome, wherein the method comprises editing a nuclear genome by the method for editing a plant genomic DNA according to the first embodiment:
a method for producing a plant having an edited plastid genome, wherein the method comprises editing a plastid genome by the method for editing a plant genomic DNA according to the first embodiment: or
a method for producing a plant having an edited mitochondrial genome, wherein the method comprises editing a mitochondrial genome by the method for editing a plant genomic DNA according to the first embodiment.

The plants according to the first, second, and third embodiments are not particularly limited, and any plants may be applied as long as they are seed plants. If daring to give some examples, examples of the plants that can be used herein may include: gramineous plants, such as rice, wheat, corn, barley, rye, and sorghum: and cruciferous plants, for example, plants belonging to genus Alyssum, genus Arabidopsis (Arabidopsis thaliana, etc.), genus Armoracia (horseradish, etc.), genus Aurinia, genus Brassica (Chinese flat cabbage, mustard green, Brassica juncea, rapeseed, Brassica rapa ssp., hagoromokanran (kale), flowering kale, cauliflower, cabbage, brussels sprouts (komochikaran), broccoli, bok choy, turnip greens mustard leaves, oilseed rape, Chinese cabbage, Japanese mustard spinach, turnip, etc.), genus Camelina, genus Capsella, genus Cardamine, genus Coronopus, genus Diplotaxis, genus Draba, genus Eruca (Rucola, etc.), genus Hesperis, genus Hirschfeldia, genus Iberis, genus Ionopsidium, genus Lepidium, genus Lobularia, genus Lunaria, genus Malcolmia, genus Matthiola, genus Nasturtium, genus Orychophragmus, genus Raphanus (Japanese radish, Raphanus sativus var. sativus, etc.), genus Rapistrum, genus Rorippa, genus Sisymbrium, genus Thlaspi, and genus Eutrema (Japanese wasabi mustard, etc.). Furthermore, other examples of the plants that can be used herein may include: solanaceous plants, such as tomato, potato, pepper, shishito pepper, and petunias: Asteraceae plants, such as sunflower and dandelion: Convolvulaceae plants, such as bindweed and sweet potato: araceous plants, such as konjak, taro, Colocasia esculenta, and Colocasia esculenta: leguminous plants, such as soybeans, adzuki beans, and green beans: cucurbitaceous plants, such as pumpkin, cucumber, and melon: and amaryllidaceous plants, such as onion, green onion, and garlic.

The disclosures of all publications cited in the present description are incorporated herein by reference in their entirety. In addition, throughout the present description, when the description includes singular terms with the articles “a,” “an,” and “the,” these terms include not only single items but also multiple items, unless otherwise clearly specified from the context.

Hereinafter, the present invention will be further described in the following examples. However, these examples are only illustrative examples of the embodiments of the present invention, and thus, are not intended to limit the scope of the present invention.

### EXAMPLES

### I. Editing of Plastid Genome

### I-1. Materials and Methods

### I-1-1. Plant Materials and Cultivation Conditions

A wild-type strain, Arabidopsis thaliana Colombia-0 strain (Col-0), and a genetically recombinant strain were cultivated at 22° C. under long-day conditions (light period: 16 hours; dark period: 8 hours). Col-0 seeds were seeded on a ½ MS medium (pH=5.7) containing Murashige-Skoog medium salt mixture (Wako, Japan) (2.3 g/L), MES (500 mg/L) and sucrose (10 g/L), and on a ½ MS medium containing Plant Preservative Mixture (Plant Cell Technology, USA) (1 mL/L), Gamborg's Vitamin Solution (Sigma-Aldrich, USA) (1 mL/L) and agar (8 g/L). One to two weeks after the seeding, the seedlings were transplanted in Jiffy-7 (Jiffy Products International B. V., Netherlands), and were then used in Agrobacterium transfection. Besides, several slow-growing T₁ plants were subjected to a stratification treatment, and were then transplanted into plant boxes each containing a ½ MS medium at 23 days after stratification (DAS) (at 23 DAS).

### I-1-2. Designing of TALE-Binding Sequences

TALE target sequences were designed using Old TALEN Targeter (https://tale-nt.cac.cornell.edu/node/add/talen-old), such that the sequences bind to both sides of a cytidine deaminase target region. A first nucleotide to be recognized needs to be on the 3′ side adjacent to T, as far as possible. The minimum length of the TALE target sequence was set to be 15 bp in order for TALE to bind in a sequence-specific manner. The TALE-binding sequences are shown below.

16S rRNA
TALE left-binding sequence:
(SEQ ID NO: 1)
5′-TAACCCAACACCTTACGGCACG-3′
TALE right-binding sequence:
(SEQ ID NO: 2)
5′-CGGACACAGGTGGTGCAT-3′
rpoC1
TALE left-binding sequence:
(SEQ ID NO: 3)
5′-TGTTGATGTTTATACCGA-3′
TALE right-binding sequence:
(SEQ ID NO: 4)
5′-TCGGAATGAATCACAAAAT-3′
psbA
TALE left-binding sequence:
(SEQ ID NO: 5)
5′-TTTCGCGTCTCTCTAA-3′
TALE right-binding sequence:
(SEQ ID NO: 6)
5′-TTAAATAAACCAAGGATTT-3′

### I-1-3. Construction of TALECD Expression Vector

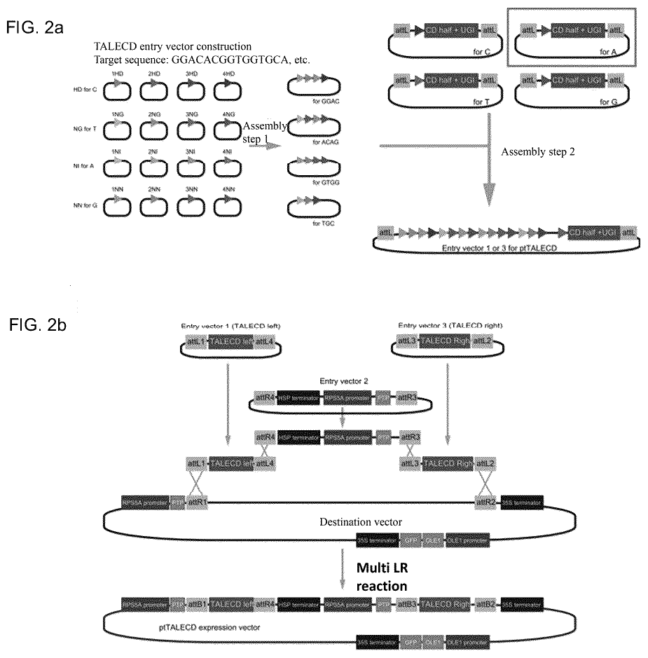

One pair of left and right ptpTALECDs (FIG. 2) incorporated into a Ti plasmid, which were for each target, were constructed using Platinum Gate assembling kit and Multisite Gateway (Thermo Fisher) according to the previously reported method for producing mitoTALENs (Kazama et al., Nature plants 5, 722-730, 2019).

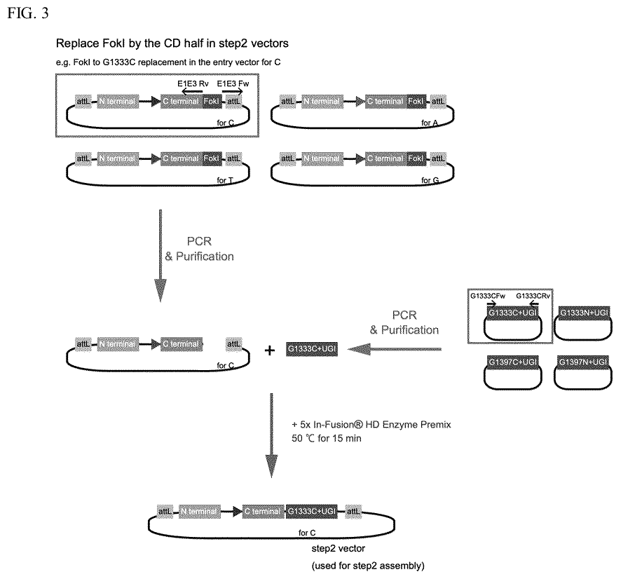

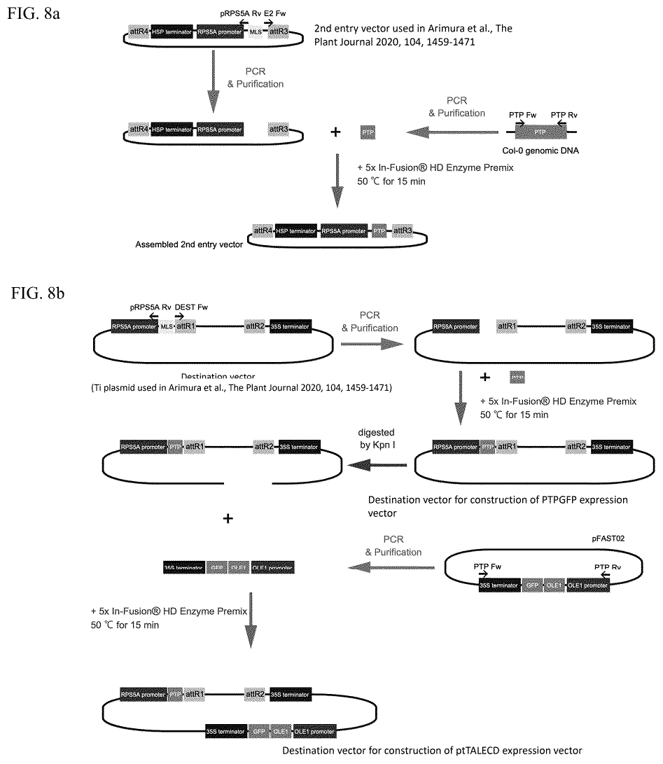

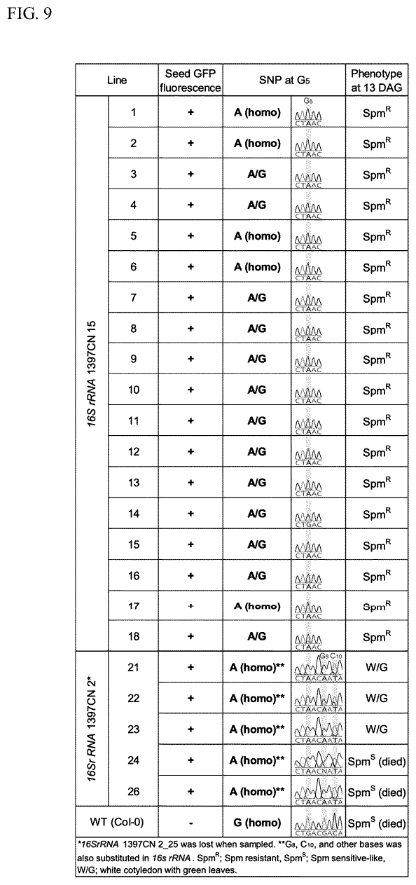

The DNA binding domains of ptpTALECDs were assembled using Platinum Gate TALEN system (Sakuma et al., Scientific reports 3, 1-8, 2013.) (FIG. 2a). The FokI coding sequences of mitoTALENs used in the previously reported assembly-step 2 had previously been replaced with CD half and UGI coding sequences, using In-Fusion HD cloning kit (TaKaRa, Japan, FIG. 3). The CD half and UGI coding sequences were designed to encode the same sequence as the amino acid sequence disclosed in Non Patent Literature 3, and were then synthesized by Eurofins Genomics (https://www.eurofinsgenomics.jp/jp/orderpages/gsy/gene-synthesis-multiple/), using codons optimized for Arabidopsis thaliana. The assembled ORFs of a 1st entry vector, a 3rd entry vector, and a 2^{nd} entry vector were incorporated into the Ti plasmid (Arimura et al., The Plant Journal 104, 1459-1471, 2020.) by a multi-LR reaction using LR Clonase™ II Plus enzyme (Thermo Fisher Scientific) (FIG. 2b). The 2^{nd} entry vector had a terminator of Arabidopsis thaliana heat shock protein (Nagaya et al., Plant and cell physiology 51, 328-332, 2010), an Arabidopsis thaliana RPS5A promoter, and the N-terminal peptide (51 amino acids) of the plastid transit peptide (PTP) of Arabidopsis thaliana RECA1 (FIG. 8a). This Ti plasmid was constructed by replacing the CaMV 35S promoter of the Gateway destination Ti plasmid pK7WG2 (Karimi et al., Trends in plant science 7, 193-195, 2002.) with the Arabidopsis thaliana RPS5A promoter (Tsutsui et al., Plant and Cell Physiology 58, 46-56 2017), and then by inserting the PTP coding sequence and proOleosin::Ole1-GFP derived from pFAST02 (http://www.inplanta.jp/pfast.html, INPLANTA INNOVATIONS INC., Japan) (FIG. 8b).

Hereafter, CD half-UGI sequences and a RecA1 PTP sequence are shown.

G1333C + UGI sequence:
(SEQ ID NO: 7)
GGTAGTCCAACTCCGTATCCGAATTACGCCAATGCAGGACATGTTGAAG
GTCAATCTGCATTGTTCATGAGGGATAACGGCATTTCTGAAGGGTTGGTG
TTCCACAACAACCCTGAAGGAACATGTGGATTTTGCGTCAACATGACAG
AAACCCTTCTCCCAGAAAACGCTAAGATGACAGTAGTTCCACCTGAAGG
TGCTATTCCTGTCAAAAGAGGTGCTACTGGTGAAACCAAGGTGTTTACT
GGGAATTCCAATTCACCCAAAAGCCCAACGAAAGGTGGGTGTAGTGGA
GGATCTACAAATCTCTCTGACATCATTGAGAAAGAGACTGGAAAGCAAC
TAGTCATTCAGGAGTCAATCCTGATGTTACCAGAGGAGGTTGAGGAAGT
GATAGGCAATAAGCCCGAAAGCGATATACTTGTTCATACTGCCTATGACG
AATCGACGGATGAGAACGTAATGCTTCTAACCTCAGATGCTCCTGAGTA
CAAACCTTGGGCGTTAGTTATCCAGGATTCCAATGGAGAGAACAAGATC
AAGATGTTG

“G1333C” is a protein consisting of the amino acids at positions 45 to 138 on the C-terminal side of the amino acid sequence of DddAₜₒₓ as set forth in SEQ ID NO: 35. In addition, UGI (Uracil Glycosylase Inhibitor) consists of the amino acid sequence as set forth in SEQ ID NO: 36, and is ligated to the “G1333C” via a linker peptide (SEQ ID NO: 37) (hereinafter, the amino acid sequence of UGI and the linker peptide are the same as those described above).

G1333N + UGI sequence:
(SEQ ID NO: 8)
GGATCTGGTAGCTATGCGTTAGGACCCTATCAGATTTCAGCTCCTCAATT
GCCTGCCTATAATGGGCAAACTGTTGGCACCTTTTACTACGTCAATGATG
CTGGAGGGTTAGAATCCAAGGTGTTCTCAAGTGGTGGTTCTGGAGGTAG
TACGAATCTTTCGGACATCATAGAGAAGGAAACTGGAAAACAGCTCGTT
ATCCAAGAGAGCATTCTCATGTTGCCAGAAGAAGTTGAAGAGGTTATAG
GCAACAAACCGGAATCTGACATTCTGGTACATACCGCTTATGATGAGTCA
ACAGATGAGAACGTCATGCTTTTGACATCTGATGCACCAGAATACAAAC
CTTGGGCACTTGTGATTCAGGATTCCAATGGTGAGAACAAGATCAAGAT
GCTA

“G1333N” is a protein consisting of the amino acids at positions 1 to 44 on the N-terminal side of the amino acid sequence of DddAₜₒₓ as set forth in SEQ ID NO: 35.

G1397C + UGI sequence:
(SEQ ID NO: 9)
GGTTCTGCGATTCCAGTTAAGAGAGGAGCTACAGGAGAAACGAAAGTC
TTTACTGGGAATTCCAATTCTCCCAAATCACCGACTAAAGGCGGATGTAG
TGGTGGTAGTACCAATCTTTCCGACATTATCGAGAAGGAAACAGGTAAA
CAACTCGTAATCCAAGAAAGCATACTGATGCTTCCTGAAGAGGTTGAAG
AGGTCATAGGGAACAAACCTGAAAGCGACATTTTGGTTCATACTGCCTA
TGATGAGTCTACAGATGAGAACGTGATGTTGCTAACCTCAGATGCACCT
GAATACAAGCCATGGGCTTTAGTGATTCAGGATTCGAATGGAGAGAACA
AGATCAAGATGCTC

“G1397C” is a protein consisting of the amino acids at positions 95 to 138 on the C-terminal side of the amino acid sequence of DddAₜₒₓ as set forth in SEQ ID NO: 35.

G1397N + UGI:
(SEQ ID NO: 10)
GGGTCTGGATCGTATGCTTTAGGACCGTATCAGATCTCAGCTCCACAATT
GCCTGCATATAACGGACAAACTGTTGGGACCTTTTACTACGTTAACGATG
CTGGTGGATTGGAGTCCAAAGTGTTCTCTTCTGGTGGCCCAACTCCATAT
CCCAATTATGCGAATGCAGGCCATGTTGAAGGTCAATCAGCCCTATTCAT
GAGAGATAACGGAATAAGTGAAGGACTGGTGTTTCACAACAATCCAGA
AGGTACTTGTGGATTTTGCGTAAACATGACTGAGACACTTCTCCCAGAA
AATGCCAAGATGACAGTTGTACCTCCTGAAGGTTCTGGTGGATCGACAA
ACCTTTCAGACATTATCGAGAAAGAGACAGGCAAACAGCTAGTGATTCA
AGAGTCCATTCTCATGCTTCCCGAAGAAGTTGAGGAAGTCATTGGGAAT
AAGCCGGAAAGTGACATACTCGTTCATACGGCTTACGATGAGAGCACGG
ATGAGAATGTCATGTTGCTTACCAGTGATGCACCTGAATACAAACCTTGG
GCTTAGTCATCCAGGACAGCAATGGTGAGAACAAGATCAAGATGCTG

“G1397N” is a protein consisting of the amino acids at positions 1 to 94 on the N-terminal side of the amino acid sequence of DddAₜₒₓ as set forth in SEQ ID NO: 35.

PTP coding sequence of RecA1:
(SEQ ID NO: 11)
ATGGATTCACAGCTAGTCTTGTCTCTGAAGCTGAATCCAAGCTTCACTCC
TCTTTCTCCTCTCTTCCCTTTCACTCCATGTTCTTCTTTTTCGCCGTCGC
TCCGGTTTTCTTCTTGCTACTCCCGCCGCCTCTATTCTCCGGTTACCGTC
TACGCCGCGAAG

“PTP” is a plastid transit peptide of Arabidopsis thaliana RECA1 (the amino acid sequence of PTP is as set forth in SEQ ID NO: 38).

Primer sequences used in vector construction are shown in the following Table 1.

TABLE 1

| Primer Name | Primer Sequence (5′to 3′) | Template |
|---|---|---|
| E1E3_Fw | TGATAACTCGAGCGATCCTC (SEQ ID NO: 12) | Step 2 entry vector containing Foki |
| E1E3_Rv | CCCCAATCCCTTTTTCACTG (SEQ ID NO: 13) | coding sequence |
| G1333CFw | AAAAAGGGATTGGGGGGTAGTCCAACTCCGTATCC | G1333C + UGI |
| | SEQ ID NO: 14) | |
| G1333CRv | TCGCTCGAGTTATCACAACATCTTGATCTTGTTCTCTCC | |
| | (SEQ ID NO: 15) | |
| G1333NFw | AAAAAGGGATTGGGGGGATCTGGTAGCTATGCGTT | G1333N + UGI |
| | (SEQ ID NO: 16) | |
| G1333NRv | TCGCTCGAGTTATCATAGCATCTTGATCTTGTTCTCACC | |
| | (SEQ ID NO: 17) | |
| G1397CFw | AAAAAGGGATTGGGGGGGTTCTGCGATTCCAGTTAAG | G1397C + UGI |
| | (SEQ ID NO: 18) | |
| G1397CRv | TCGCTCGAGTTATCAGAGCATCTTGATCTTGTTCTC | |
| | (SEQ ID NO: 19) | |
| G1397NFw | AAAAAGGGATTGGGGGGGTCTGGATCGTATGCTTT | G1397N + UGI |
| | (SEQ ID NO: 20) | |
| G1397NRv | TCGCTCGAGTTATCACAGCATCTTGATCTTGTTCTC | |
| | (SEQ ID NO: 21) | |
| PTPFw | ATGGATTCACAGCTAGTCTTGTCTC (SEQ ID NO: 22) | Col-0 genomic ONA |
| PTPRf | CTTCGCGGCGTAGACGGTAAC (SEQ ID NO: 23) | |
| E2 Fw | ATGGATTCACAGCTAGTCTTGTCTC (SEQ ID NO: 24) | 2nd entry vector |
| pRPSSA Rv | GTCTACGCCGCGAAGACAACTTTGTATAATAAAGTTGAACG | 2nd entry vector and destination |
| | (SEQ ID NO: 25) | vector |
| DEST Fw | GTCTACGCCGCGAAGGCTGTGATATCACAAGTTTG | Destination vector |
| | (SEQ ID NO: 26) | |

I-1-4. Transformation of Plants and Screening of Transformants

Col-0 was transformed by a floral dip method (Clough et al., The Plant Journal 16, 735-743, 1998.) with the Agrobacterium tumefaciens strain C58C1 retaining one of the aforementioned transformation vectors. First, transgenic T₁ seeds were selected using fluorescence from GFP as an indicator. GFP-positive seeds were seeded on a ½ MS medium containing 125 mg/L Claforan. On the other hand, GFP-negative seeds were seeded on a ½ MS medium containing 50 mg/L kanamycin and 125 mg/L Claforan.

### I-1-5. Sanger Sequencing and Next-Generation Sequencing (NGS)

Total DNA was extracted from the second true leaf of the selected seedlings, using the Maxwell (registered trademark) RSC Plant DNA Kit (Promega, USA). For genotyping of transgenic strains, the plastid DNA sequence regions around the cytidine deaminase target sequences were amplified using the following primer sets corresponding to the target genes. In order to detect substitution of the target nucleotide, the nucleotide sequences of the purified PCR products were determined by the Sanger method.

16S rRNA
(SEQ ID NO: 27)
Forward primer: 5′-GGTTCCAAACTCAACGGTGG-3′
(SEQ ID NO: 28)
Reverse primer: 5′-TAGGGGCAGAGGGAATTTCC-3′
psbA
(SEQ ID NO: 29)
Forward primer: 5′-GGTATTATTTTAGTGGCCCA-3′
(SEQ ID NO: 30)
Reverse primer: 5′-GCCTGTGATAATAGGAAAGC-3′
rpoC
(SEQ ID NO: 31)
Forward primer: 5′- AGACGGTTTTCAGTGCTAGT-3′
(SEQ ID NO: 32)
Reverse primer: 5′- TTTGGGGAGGGGTTTTTTAC-3′

Using all DNA sequence data, single nucleotide polymorphisms (SNPs) in the plastid and mitochondrial genomes were determined. First, preparation of a PE library using Nextera XT DNA library Prep Kit (Illumina) was entrusted to Macrogen Japan, and sequencing was then carried out using Illumina NovaSeq 6000 platform. Sequence reads at the 150 bp paired end were analyzed using Geneious prime (Biomatters Ltd). Sequence reads were attached to an Arabidopsis thaliana chloroplast genome sequence, and sequences detected as SNPs with a reference chloroplast genome sequence in 50% or more of the reads are shown in the following Table 2.

TABLE 2
Mutation determined in plastid genome of [symbol] plant, compared
with reference genome [symbol] (Mutation percentage: >50%)

| | Gene or region | Position | [symbol] | [symbol] | [symbol] | Remarks |
|---|---|---|---|---|---|---|
| Wt (Col-0) | — | — | — | — | — | No [symbol] found in 3 analyzed plants |
| 16SrRNA 1397CN1** | 16SrRNA | IR | [symbol] | TC → TT | n.a. | Mutation predicted to cause |
| | | | | | | biological effects |
| | | | [symbol] | TC → TT | n.a. | |
| | | | [symbol] | GA → AA | n.a. | |
| | ref2 | IR | [symbol] | GA → AA | Gly → Glu | Essential gene, ATPase-related, |
| | | | | | | functions unknown |
| | rps14-t [symbol] fM | LSC | [symbol] | TC → TT | n.a. | Intergenic region |
| | p [symbol] A-p [symbol] | LSC | [symbol] | TC → TT | n.a. | Intergenic region |
| | [symbol] A- [symbol] A | IR | [symbol] | TC → TT | n.a. | Intron |
| 16SrRNA 1397CN2 | 16SrRNA | IR | [symbol] | TC → TT | n.a. | Mutation predicted to cause |
| | | | | | | biological effects, no off-target |
| 16SrRNA 1397CN7 | 16SrRNA | IR | [symbol] | TC → TT | n.a. | Mutation predicted to cause |
| | | | | | | biological effects, no off-target |
| 16SrRNA 1397CN8 | 16SrRNA | IR | [symbol] | TC → TT | n.a. | Mutation predicted to cause |
| | | | | | | biological effects, no off-target |
| 16SrRNA 1397CN12 | 16SrRNA | IR | [symbol] | TC → TT | n.a. | Mutation predicted to cause |
| | | | | | | biological effects, no off-target |
| 16SrRNA 1397CN16 | 16SrRNA | IR | [symbol] | TC → TT | n.a. | Mutation predicted to cause |
| | | | | | | biological effects, no off-target |
| 16SrRNA 1397NC1 | 16SrRNA | IR | [symbol] | AC → AT | n.a. | Mutation of target region, no |
| | | | | | | off-target |
| | 16SrRNA | IR | [symbol] | TC → TT | n.a. | Mutation predicted to cause |
| | | | | | | biological effects, no off-target |
| 16SrRNA 1397NC2 | 16SrRNA | IR | [symbol] | AC → AT | n.a. | Mutation of target region, no |
| | | | | | | off-target |
| | 16SrRNA | IR | [symbol] | TC → TT | n.a. | Mutation predicted to cause |
| | | | | | | biological effects, no off-target |
| 16SrRNA 1397NC3 | 16SrRNA | IR | [symbol] | AC → AT | n.a. | Mutation of target region, no |
| | | | | | | off-target |

*Position of intergenic mutation from first [symbol] of the gene
IR: [symbol] region [symbol]
LSC: [symbol] ong single copy region [symbol]
n.a.: Not applicable
**Single [symbol] and withered and died around [symbol] [symbol]
indicates data missing or illegible when filed

### I-1-6. Genotyping of T₂ Plants

T₂ seeds obtained from T₁ plants corresponding to individual target genes were seeded on a ½ MS medium. Genotyping of 16S rRNA in the cotyledons of 7 DAS or 13 DAS seedlings was performed as in the case of the T₁ plants. PCR for GFP was performed using the following primers.

Forward primer:
(SEQ ID NO: 33)
5′- GGTGATATCCCGCGGATGGTGAGCAAGGGCGAGGA-3′
Reverse primer:
(SEQ ID NO: 34)
5′- ACGTAACATGCCGGGCTTGTACAGCTCGTCCATGC-3′

### I-1-7. Screening of Spectinomycin-Resistant Plants

At 11 DAS and 23 DAS, T₂ seeds derived from the T₁ plants, in which C₅ of 16S rRNA was homoplasmically substituted, were seeded on a ½ MS medium containing 0, 10 or 50 mg/L spectinomycin. The phenotypes of germinated cotyledons were observed at 8 DAS.

### I-1-8. Image Processing

Plant images were taken with iPhone (registered trademark) Xs (Apple Inc., US) and LEICA MC 170 HD (Leica, Germany). Gel images were taken with a ChemiDoc™ MP Imaging System (BIORAD, USA). Then, the images were processed with Adobe Photoshop 2021 (Adobe, USA).

### I-2. Results

### I-2-1. TALECD Expression Vector

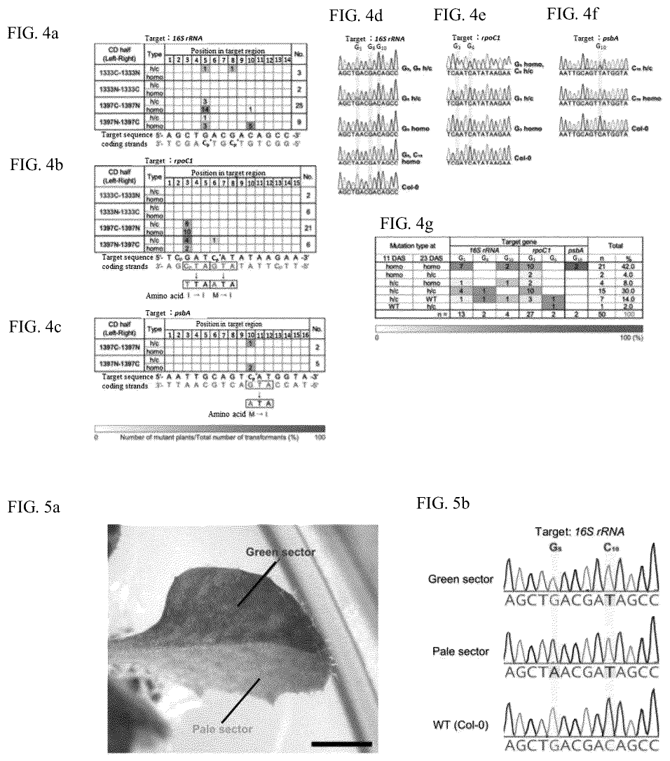

The amino acid sequence of DddAₜₒₓ as set forth in SEQ ID NO: 35 was divided between the 44th and 45th amino acids, or between the 94th and 95th amino acids, and the N-terminal or C-terminal side was linked to the C-terminus of a platinum TALE DNA-binding domain (Sakuma et al., Scientific reports 3, 1-8, 2013.) (pTALECD, FIG. 1a). A plastid targeting signal peptide (PTP) of an Arabidopsis thaliana RECA1 protein (FIG. 1b) was linked to the N-terminal side of pTALECD. In addition, in order to inhibit the hydrolysis of uracil (U) generated by cytidine deaminase, a uracil glycosylase inhibitor (UGI) (Non Patent Literature 3) was linked thereto (FIG. 1b). The nucleotide sequences of DddAₜₒₓ (CD) and UGI were optimized to the codon usage frequency of Arabidopsis thaliana. A PTP-pTALECD-UGI (ptpTALECD) pair (a pair including the N-terminal side and C-terminal side of CD) was allowed to express under an RPS5A promoter (Arimura et al., The Plant Journal 104, 1459-1471, 2020) using a single plant transformation vector (FIG. 1b). By modifying the method disclosed in the previous report (Kazama et al., Nature plants 5, 722-730, 2019), an assembly system for easily constructing a tandem ptpTALECD expression vector for each target sequence on a Ti plasmid was established (FIGS. 2a and b). In the present example, FokI in the vector used in the method disclosed in the previous report was substituted with CD-UGI (FIG. 3). The constructed vector was introduced into the nucleus of Arabidopsis thaliana by the floral dip method, and an attempt was made to substitute C/G with T/A in the three regions of the plastid genome, namely, the 16S rRNA gene region (FIG. 4a), the rpoC1 region (FIG. 4b), and the psbA region (FIG. 4c).

As described above, 12 types of ptpTALECD expression vectors (expression vectors targeting the three regions by four CD half combinations (see FIG. 1a)) were constructed.

Each expression vector was introduced into Arabidopsis thaliana, and at 23 DAS, the target region of T₁ was sequenced by the Sanger method. Only the constructs, in which T₁ was obtained, are shown in FIGS. 4a, b, and c. The results that the C/G pair was substituted with T/A in all of the three target regions were confirmed in multiple T₁ constructs (FIGS. 4a-f). In addition to the heteroplasmically substituted strains or chimerically substituted strains (h/c: FIGS. 4a-f), surprisingly, a large number of strains, in which the target regions were homoplasmically substituted (homo), were observed. Not all C/G pairs in the target regions were substituted, and the substituted C/G pairs were biased in all of the three regions (FIGS. 4a-c). The homoplasmically substituted nucleotide in the three regions was C in (5′)TC(3′), which was assumed to be easily mutated according to Mok et al. (Non Patent Literature 3) (FIGS. 4a-c). Meanwhile, C in the (5′)AC(3′) of the 16S rRNA gene was also homoplasmically substituted (FIG. 4a).

In order to examine the stability of mutations in the growth process of individual plants, the nucleotide sequences of total DNAs extracted from the newborn leaves of T₁ plants at 11 DAS and 23 DAS (or from the cotyledons of slow-growing plants at 11 DAS) were examined. At 11 DAS and 23 DAS, among plants having a nucleotide mutation in the target region, several plants retained the mutant nucleotide in a heteroplasmic or chimeric (h/c) form at both time points (30.0% of all plants, 15/50, FIG. 4g). In addition, other plants had a different mutation state at both time points (e.g., homo (homoplasmic mutation) became h/c in 4.0% of all plants, 2/50: h/c became a wild type in 14.0% of all plants, 7/50: h/c became homo in 8.0% of all plants, 4/50; and a wild type became h/c in 2.0% of all plants, 1/50) (FIG. 4g). Many of the remaining plants retained the mutant nucleotide in a homoplasmic state at both time points (42.0%, 21/50, FIG. 4g). Interestingly, in the cotyledons of T₁ plants (16S rRNA 1397NC3), a wild-type-like green portion and a light-colored portion were present, and a mutation percentage was different in Cp* (cytosine predicted to cause biological effects) in 16S rRNA in each region (FIGS. 5a and b). Surprisingly, most of the homoplasmically substituted nucleotides at 11 DAS remained to be homoplasmically substituted even at 23 DAS (91.3%, 21/23). These results suggest that the target nucleotide of T₁ transformed with the ptpTALECD expression vector be homoplasmically substituted at a high frequency, and that the mutation be stably maintained throughout the growth process.

Subsequently, the off-target effect of ptpTALECD (substitution of non-target nucleotides) in the maternally inherited plastid and mitochondrial genomes was examined (the above Table 2). The total genome sequences of 14 T₁ plants were determined (Novaseq, Illumina). In the 13 plants, most of the target nucleotides C were homoplasmically substituted with T (16S rRNA 1397C-1397N (1397CN) line 2, line 7, line 8, line 12, line 16, 1397N-1397C (1397NC) line 1, line 2, line 3: psbA 1397C-1397N (1397CN) line 6, 1397N-1397C (1397NC) line 1, line 5: and rpoC1 1397C-1397N(1397CN) line 16), while one remaining target (rpoC1 1397C-1397N (1397CN) line 3: see FIGS. 4a-c) was substituted, heteroplasmically or chimerically. The plastid SNPs in which 50% or more of the reads are different from the reference genome in at least one T₁ plant are shown in Table 2. Mutations overlapped in the repeated sequences of the plastid genome were counted as one mutation. It was confirmed that most of the target nucleotides in the 13 plants were homoplasmically substituted. It was confirmed that the nucleotides in the remaining one plant were heteroplasmically or chimerically substituted (Table 2). Major off-target point mutations (substitution frequency>50%) were found in six locations in 16S TRNA 1397C-1397N (1397CN) line 1, while no off-target point mutations were detected in the other lines (Table 2). The 16S rRNA 1397CN line 1 withered and died at 23 DAS, without producing true leaves. Regarding the mitochondrial genome, no significant off-target mutations were detected in the mitochondrial genomes of all of the 14 plants including 16S rRNA 1397CN line 1. These results demonstrate that ptpTALECD only rarely introduces an off-target point mutation in the genomes of organelle, and specifically and homoplasmically substitutes the C/G in the target region with T/A.

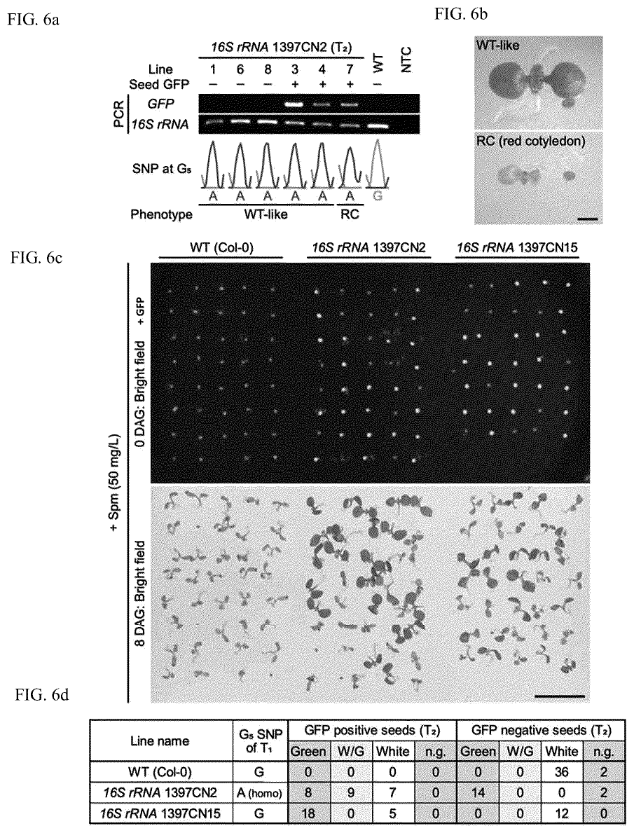

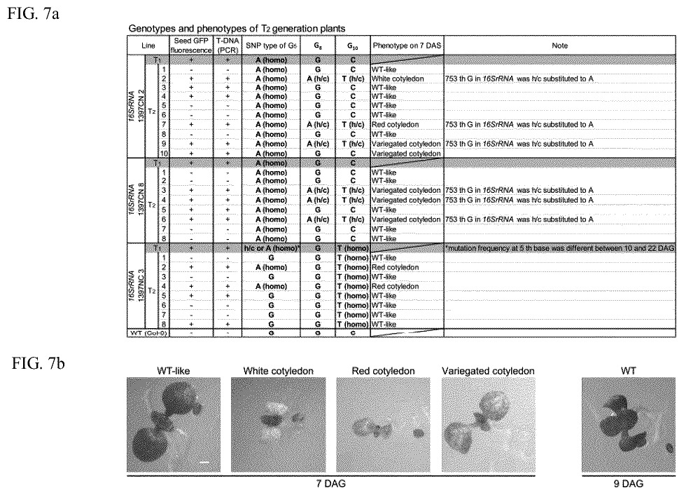

T₁ plants, which were transformed with the 16S rRNA-targeted ptpTALECD vector and in which the first Cp*(G₅) and/or C₁₀ were homoplasmically substituted, were all fertile, except for one plant (16S rRNA 1397C-1397N line 1). In order to examine whether or not the C to T substitution mutation is inherited by progenies, the genotyping of T₂ plants of these three strains (16S rRNA 1397C-1397N line 2, line 8 and 1397N-1397C line 3) was performed (FIG. 6a and FIG. 7a). Based on the results of seed-specific GFP (green fluorescent protein) derived from Ole1 pro::Ole1-GFP13 on T-DNA (FIG. 1b) and GFP PCR (FIG. 6a), the T₂ plants were classified into T-DNA transgene-free plants (null segregants) and transgenic plants. All of the T₂ plants stably retained the homoplasmic mutation (FIG. 6a and FIG. 7a). Interestingly, the cotyledons of several T₂ plants were white, red or mottled (FIG. 6b and FIG. 7b), and were different from the phenotypes of their parents. Such plants were all GFP-positive (FIG. 6a and FIG. 7a), and many of them (8 out of 9 plants) had other mutations up to 400 bp examined in the 16S rRNA sequence (FIG. 7a). Since it had been reported that the RPS5A promoter used for ptpTALECD expression is significantly expressed in oocytes, it is conceived that de novo mutations may have occurred in the early developmental stages of the T₂ plants, resulting in abnormal cotyledons. Differing from these T₂ plants, the T₂ plants as null segregants did not exhibit the additional phenotypes as described above, and retained the target mutation. The aforementioned results demonstrate that the plastid genome having an artificially introduced point mutation is stably inherited by progenies, and further that it is independent from the inheritance of nuclear T-DNA. Furthermore, the aforementioned results also demonstrate that null segregants having a targeted point mutation in the plastid genome can be successfully established.

G₅ of the 16S rRNA gene corresponds to G, which is predicted to cause biological effects on E. coli 16S rRNA, and the substitution mutation of G in this E. coli 16S rRNA is known to confer spectinomycin resistance (Spm^{r}). T₂ seeds collected from T₁ plants (16S rRNA 1397C-1397N line 2) in which G₅ was homoplasmically substituted with A were seeded on a spectinomycin-containing medium. Regardless of the presence or absence of GFP fluorescence from the seeds, many of the seedlings germinated from these seeds showed spectinomycin resistance (FIG. 6c). However, several T₂ plants derived from 16S rRNA 1397C-1397N line 2 showed spectinomycin-sensitive (Spm^{s})-like phenotypes (white, undeveloped plants with purple cotyledons, FIG. 6c). All of these spectinomycin-sensitive undeveloped plants were germinated from GFP-positive seeds (FIG. 6c), and many of them (5 out of 5 plants, FIG. 9) had multiple de novo mutations in the 16S rRNA gene. These results suggest that de novo mutations cause 16S rRNA dysfunction, resulting in spectinomycin-sensitive-like phenotypes (wherein spectinomycin is a drug that inhibits 16S rRNA). Surprisingly, several progenies of T₁ plants (16S rRNA 1397C-1397N line 15) that did not have a mutation in G₅ exhibited spectinomycin resistance. These progenies (18 plants) were germinated from GFP-positive seeds (FIG. 6c). In 5 of these progenies, G₅ was homoplasmically substituted with A, and in the remaining 13 progenies, many G₅s were substituted with A (FIG. 9). These results suggest that the inherited T-DNA caused de novo mutations to G₅. These results suggest that the homoplasmic mutation of G₅ to A confers spectinomycin resistance to Arabidopsis thaliana. Furthermore, the results that GFP-negative T₂ plants show spectinomycin-resistant or spectinomycin-sensitive phenotypes predicted by SNPs of G₅ in T₁ plants suggest that null-isolated T₂ plants are likely to inherit the mutations from their parental plants.

The above-described results demonstrated that ptpTALECD can introduce a target region-specific and homoplasmic C to T mutation into the plastid genome of Arabidopsis thaliana, and that this mutation is stably inherited by the offspring seeds (probably, following a maternal mode of inheritance).

### II. Editing of Mitochondrial Genome

### II-1. Materials and Methods

### II-1-1. Plant Materials, Cultivation Conditions, Transformation, and Screening of Transformant

Arabidopsis thaliana Col-0, otp87 (a homozygous T-DNA insertion line, GK-073C06-011724), and transformants were cultivated at 22° C. under long day conditions (a light period of 16 hours, and a dark period of 8 hours). The Col-0 seeds were seeded on a ½ MS-Agar plate (Non Patent Literature 7). Seedlings with 2 to 3 weeks old were transferred to Jiffy-7 (Jiffy Products International), and were then infected with Agrobacterium. Mature plants of Col-0 and otp87 were transformed by the floral dip method (Clough et al., The Plant Journal 16, 735-743, 1998). The obtained T₁ seeds were selected based on the seed-specific GFP fluorescence (Non Patent Literature 7: Shimada et al., Plant J. 61, 519-528, 2010). These T₁ seeds were seeded on the above-described medium containing 125 mg/L Claforan. T₁ plants were transplanted to Jiffy-7 at 23 DAS. OTP87 seeds (GABI_073C06) were obtained from ABRC Stock Center. The homozygosity of OTP87 T-DNA insertion in the plants was confirmed by PCR (Hammani et al., J. Biol. Chem. 286, 21361-21371, 2011).

### II-1-2. Designing of TALE-Binding Sequences and Construction of Vectors

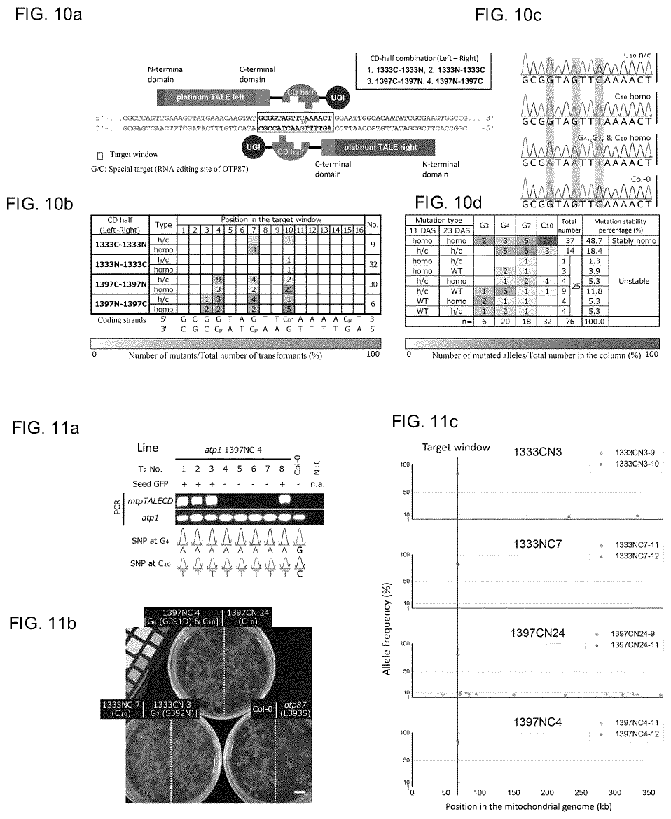

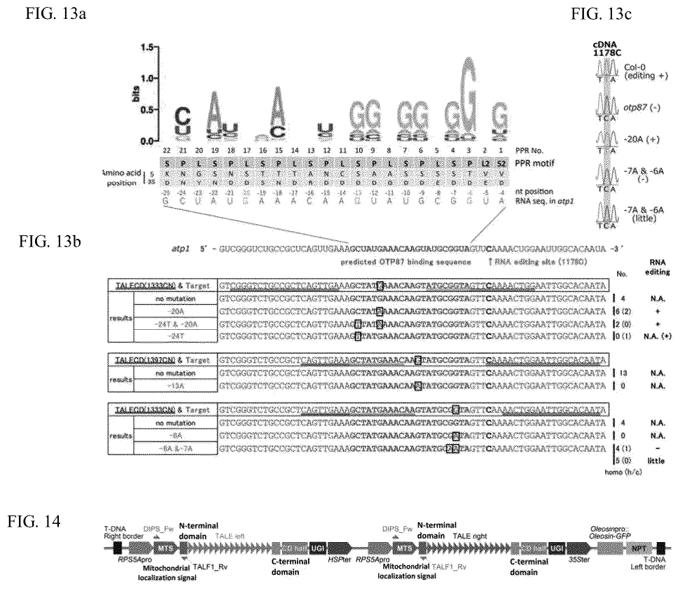

TALE-binding sequences are shown in FIG. 10a and FIG. 13b. The nucleotide recognized by TALE was located adjacent to the 3′ side of thymine, and its length was set to be about 20 bp. The length of the target window (16 bp) and the position of the special target cytosine (C10) were set based on the successful example disclosed in a previous report (Nakazato et al., Nature Plants 7, 906-913, 2021). A binary vector expressing mtpTALECD was constructed using the Platinum Gate TALEN system (Sakuma et al., Scientific reports 3, 1-8, 2013) and the multisite gateway (Thermo Fisher) in almost the same manner as the previous report (Nakazato et al., Nature Plants 7, 906-913, 2021). However, with regard to a destination vector and an entry vector used in the multi-LR reaction, those having mitochondrial localization signals, instead of chloroplast transition signals, were used.

II-1-3. Genotyping of T₁ and T₂ Plants

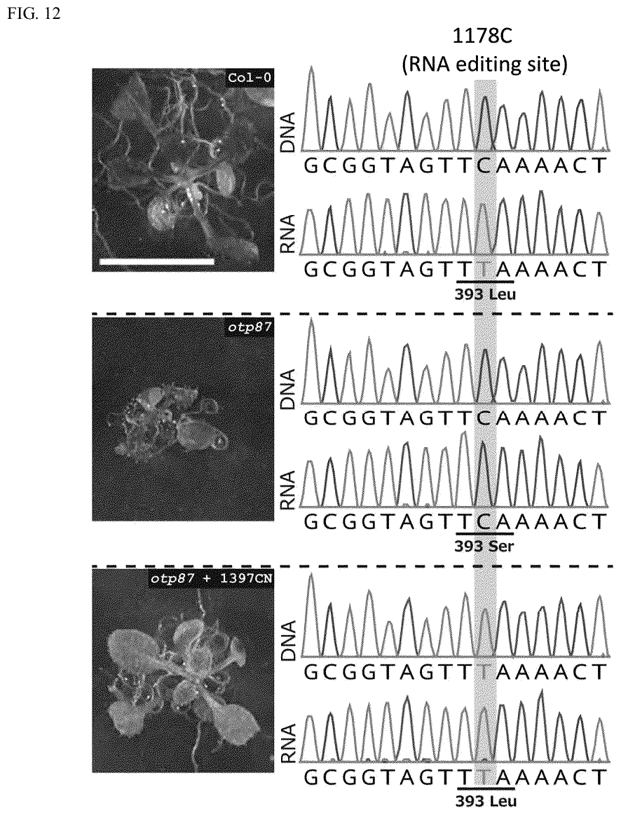

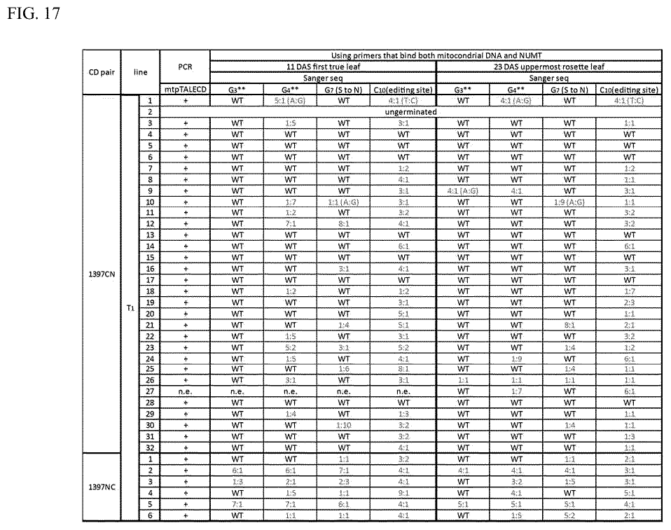

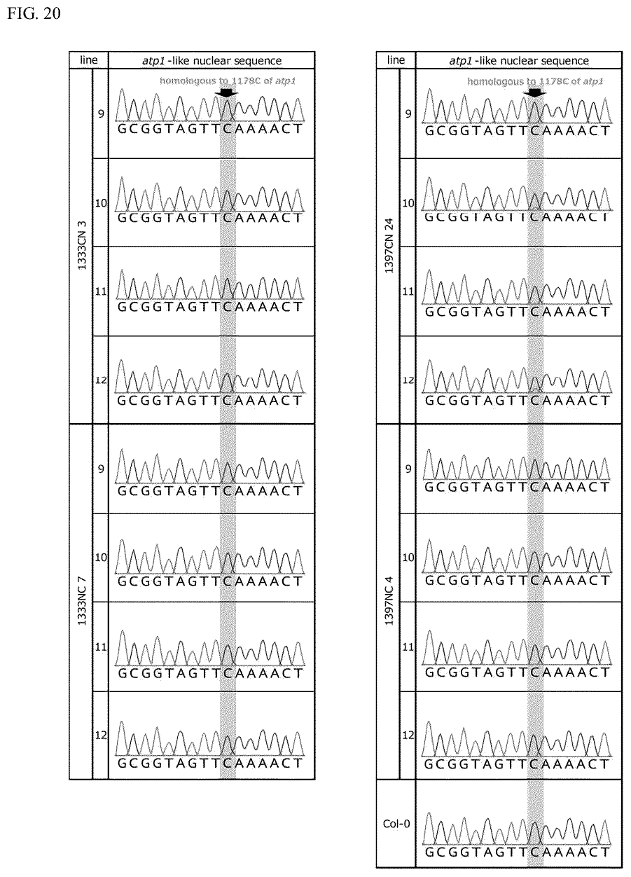

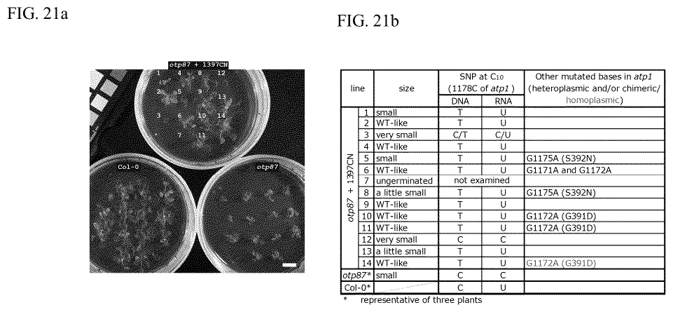

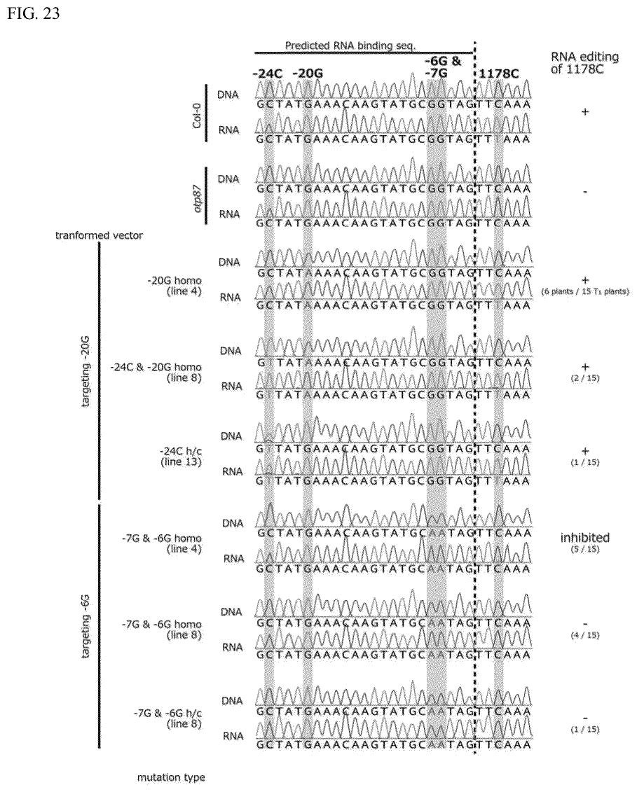

PCR for Sanger sequencing (FIG. 10, FIG. 11, FIG. 15, FIG. 16, FIG. 17, and FIG. 20) was performed employing KOD One PCR Master Mix (Toyobo Co., Ltd.), using DNA roughly extracted from true leaves or cotyledons, according to standard protocols. Nucleic acid templates used in the PCR for Sanger sequencing (FIG. 12, FIG. 13, FIG. 21, and FIG. 23) were extracted employing the Maxwell RSC Plant RNA Kit (Promega), without using DNase I included therewith. DNA in the extracted nucleic acids was decomposed with Deoxyribonuclease (RT Grade) for Heat Stop (Nippon Gene) to prepare RNA templates for RT-PCR. The RT-PCR was performed using PrimeScript™ II High Fidelity One Step RT-PCR Kit (TaKaRa). A portion of the mtpTALECD reading frame was amplified with primers, and a transformant was identified. Sequences around the target windows of mitochondrial DNA and cDNA and their homologous sequences in the nuclear DNA were amplified. The purified PCR products were read by Sanger sequencing, and the data were then analyzed by Geneious Prime (v. 2021. 2.2).

Total DNA for NGS was extracted from mature leaves using the DNeasy Plant Pro Kit (QIAGEN). A paired-end library of 11 samples using VAHTS Universal Pro DNA Library Prep Kit for Illumina (Vazyme, China) and the sequencing of 5G base/sample using Illumina NovaSeq 6000 platform were performed at GENEWIZ Japan. Whole genome sequence data for performing SNP calling were obtained for 3 samples of wild-type plants and 8 samples of T₂ plants (2 samples from each of 4 strains). As a pre-treatment of the analysis, low-quality sequences and adapter sequences contained in the reads were trimmed using PEAT [v1.2.4 (Li et al., BMC Bioinformatics, (BioMed Central, 2015), pp. 1-11)]. The paired-end reads of each strain were mapped to reference sequences (mitochondrial genome BK010421.1 and chloroplast genome AP000423.1) in a single-end mode, using BWA (v 0.7.12) (Durbin, Bioinformatics 25, 1754-1760, 2009). Inappropriate map reads having a sequence identity of 97% or less or an alignment coverage percentage of 80% or less were eliminated using a filter. SNPs were called with the samtools mpileup command (-uf -d 50000 -L 2000) and the bcftools call command (-m -A -P 0.1 (Li et al., Bioinformatics 25, 207-2079, 2009)). Finally, SNPs with (AF of T₁ sample)−(average AF of 3 wild-type plants)≥0.05 were detected as off-target SNP candidates by allele frequency (AF) calculated by the bcftools, and many artifact SNPs derived from chloroplast genome sequences similar to those in NUMT and mitochondrial genomes were eliminated (FIG. 11c).

### II-1-4. Prediction of PPR-Binding Sequences

In order to predict the binding site of OTP87 in atp1, a PPR code was used (Takanaka et al., PLos one 8 e65343 2013: Yan et al., Nucleic acids research 4, 3728-3738, 2019). In this code, the combination of two important amino acid residues at positions 5 and 35 of each PPR repeat was used to calculate which nucleotides each PPR repeat was likely to recognize. The binding probability of each motif was depicted in the weblog (http://weblogo.berkeley.edu/) shown in FIG. 13a.

### II-1-5. Image Processing

The photographs of plants were taken with a digital camera (OLYMPUS OM-D E-M5) and were then processed with Adobe Photoshop 2021.

### II-2. Results

### II-2-1. Targeted Single Nucleotide Substitution of Atp1

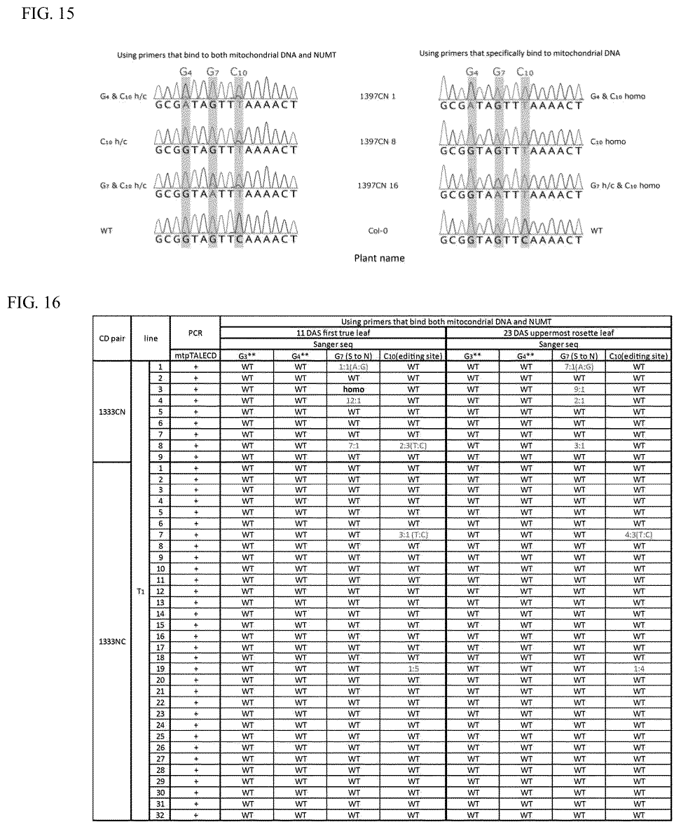

The base pair, atp1-1178C, which corresponded to the RNA editing site of mitochondrial ATPase subunit 1 (atp1), was selected as a target for nucleotide editing. In wild-type plants, this C is post-transcriptionally converted to U on the RNA and is then translated. Accordingly, when evaluating the efficiency of single nucleotide substitution and its heritability, the substitution of C:G to T:A is not considered to have adverse effects on the plants. For the substitution of this target nucleotide, 4 types of vectors containing a cytidine deaminase (CD) domain that is located at the C-terminus of a Burkholderia cenocepacia DddA protein (1,427 amino acids: Non Patent Literature 6) were produced. As in the previous reports (Non Patent Literature 6: Non Patent Literature 7: Nakazato et al., Nat. Plants 7, 906-913 2021: and Lee et al., Nat. Commun. 12, 1-6 2021), the coding sequence of the CD domain was divided at the nucleotide immediately after the codon of Gly 1333 or Gly 1397. The sequences (N- and C-terminal sides) of the divided CD halve were each fused with the 3′ side of the DNA-binding domain sequence (hereafter referred to as pTALE) of platinum TALEN (Sakuma et al., Sci. Rep. 3 1-8, 2013) that recognizes at maximum 21 nucleotides. In order to prevent the removal of uracil generated from cytosine, the sequence of pTALE-CD was fused with the 5′ side of the sequence of UGI (Non Patent Literature 6: and Mol et al., Cell 82, 701-708, 1995, pTALE-CD-UGI). The nucleotide sequences of CD and UGI are the same as those in the previous report (Nakazato et al., Nat. Plants 7, 906-913, 2021), and were optimized for the codon usage in Arabidopsis thaliana. The mitochondrial target signal sequence of the Arabidopsis thaliana ATPase delta prime subunit (Arimura et al., Plant J. 104, 1459-1471, 2020) was linked to the 5′ side of pTALE-CD-UGI (mtpTALECD: FIG. 14). Cassettes each expressing a pair of mtpTALECDs were constructed in tandem in a single binary vector. Each mtpTALECD was placed under the control of the Arabidopsis thaliana RPS5A promoter (FIG. 14), which had been used for highly efficient genome editing of Arabidopsis thaliana (Arimura et al., Plant J. 104m 1459-1471, 2020: Nakazato et al., Nat. Plants 7, 906-913, 2021; and Tsutsui et al., Plant Cell Physiol. 58, 46-56, 2017). Four binary vectors, which were named as 1333C-1333N (abbreviated as 1333CN; this name means that the C-terminal half of the CD domain divided by Gly 1333 is fused with the left TALE domain, and the N-terminal half thereof is fused with the right TALE domain), 1333N-1333C (1333NC), 1397C-1397N (1397CN), and 1397N-1397C (1397NC), were constructed (FIG. 10a).

In order to substitute the target C:G pair of the mitochondrial genome with a T:A pair, the nuclear genome of Arabidopsis thaliana was transformed with each vector by the floral dip method (Clough et al., Plant J. 16, 735-743, 1998). Total DNA from the leaves of T₁ transformants was amplified by PCR, and the nucleotide sequences of the PCR products were determined by the Sanger method. Among the 78 T₁-transformed plants examined (the number of transformants obtained with all of the four vectors), 36 plants had a substitution of C:G with T:A in the target window (FIG. 16 and FIG. 17). The plant nuclear genome often contains a large sequence fragment having high homology to mitochondrial DNA, which is called nuclear mitochondrial DNA or NUMT (Noutsos et al., Genome Res. 15, 616-628, 2005: and Zhang et al., Int. J. Mol. Sci. 21, 707, 2020). In the process of decoding a nucleotide sequence, it was found that a nuclear sequence (At2g07698) that was almost identical to atp1 as a part of NUMT on chromosome 2 of Arabidopsis thaliana Col-0 was amplified (Noutsos et al., Genome Res. 15, 616-628, 2005). Hence, in order to avoid amplification of the NUMT sequence, primers for specifically amplifying the mitochondrial DNA were newly designed and were then used in subsequent analyses.

The T₁ plants, in which a mutation had been detected by the first genotyping, were subjected to genotyping again using new primers.

In many transformants, the nucleotides in the target window appeared to be homoplasmically substituted (FIGS. 10B and C). In addition to the mutation of the target C at position 10, G₅ at positions 3, 4 and 7 in the target window were also substituted in some T₁ plants. Most of the converted Cs were on the 3′ side of T or A, as previously reported (FIG. 10b). The nucleotide substitution activity and the preference of the positions of the substituted nucleotides in the target window were different among the four vectors, and the most frequently homoplasmically substituted C in the target window was the 10th C in the case of the vector 1397C-1397N (1397CN: FIG. 10b). As a result, at both time points of 11 days and 23 days after the stratification treatment for promotion of germination (days after stratification, DAS), five mitochondrial mutant plants, in which only the true target nucleotide (10th C) was substituted in the target window, were obtained.

In order to examine whether the type of the introduced mutation is changed during the developmental process of a plant, regarding each transformant, the sequences of PCR fragments obtained using total DNAs of different leaves at 11 DAS and 23 DAS as templates were determined by the Sanger method, and the types of mutations were then examined. A total of 76 mutant nucleotides were detected on at least one of these days (FIG. 10d). Of these, 14 nucleotides were heteroplasmically or chimerically (h/c: i.e., not homoplasmically) substituted on both days, and 25 nucleotides were substituted in different ways on both days (see FIG. 10d for the number of nucleotides substituted in each type and the percentage thereof). The remaining 37 nucleotides, which accounted for about half of the mutant nucleotides detected, were homoplasmically substituted on both days [48.7% (37/76), FIG. 10d]. These results demonstrate that the C:G pair in the target window is efficiently substituted with T:A by mtpTALECD, and that there are transformants in which homoplasmic mutations are stably detected in the leaves in the two time points even in the T₁ generation.

### II-2-2. Inheritance of Introduced Mutations to Seed Progenies

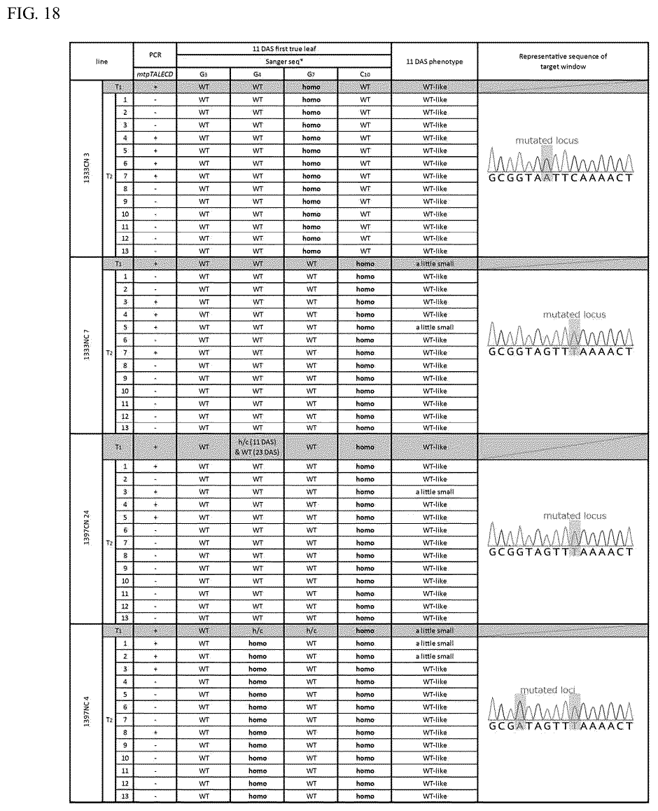

In order to confirm whether or not the introduced mutations are inherited in the seed progenies, regarding each of the 4 T₁ plants in which the C:G pair in the target window was homoplasmically substituted, T₂ progenies of 13 plants were subjected to genotyping. All of the examined T₂ plants inherited the parental homoplasmic mutation, regardless of whether they carried a mtpTALECD gene in the nucleus thereof (FIG. 11a and FIG. 18). This indicates that the homoplasmic mutation of the mitochondrial genome introduced by mtpTALECD was stably inherited in the seed progenies. Regarding each of the 4 lines, progenies that did not have the mtpTALECD gene grew as well as wild-type plants, even if they carried two different mutations causing amino acid substitution [G391D and S392N (FIG. 11b)]. Some of the nucleotides that were heteroplasmically or chimerically mutated in the T₁ generation were observed to have uniform genotypes even in the T₂ generation (FIG. 18).

### II-2-3. Off-Target Mutation on Mitochondrial Genome

In order to examine the off-target effects of mtpTALECD on the mitochondrial genome, T₂ plants (FIG. 18), which had already been confirmed to inherit the parental homoplasmic mutation generated in the target window, were measured in terms of SNP frequency. The positions and frequencies of line-specific mutant SNPs that are different from the reference sequence (BK010421.1) are shown by dots in FIG. 2C. These data demonstrate that the frequency of off-target mutations outside the target window is 10% or less of the mitochondrial DNA copies in each plant.

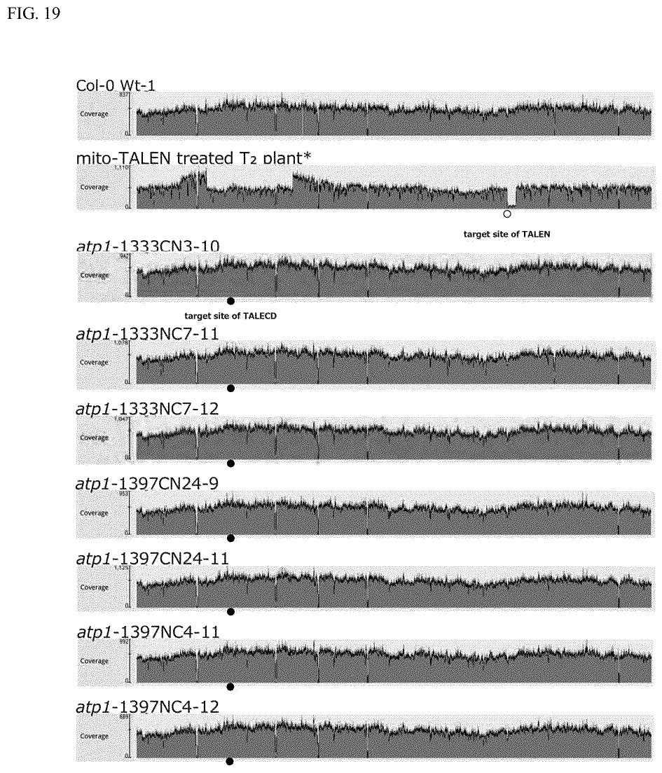

In these 8 plants, the coverage pattern of the entire mitochondrial genome was very similar to the coverage pattern of wild-type plants (FIG. 19). In addition, there were observed no findings regarding structural changes in the mitochondrial genome, such as deletions, sequence rearrangements, and generation of new repeat sequences, which had been observed in the previous studies using mitoTALEN (Kazama et al., Nat. Plants 5, 722-730, 2019; and Arimura et al., Plant J. 104, 1459-1471, 2020).

About 20% of the reads at the position of SNPs in the target window did not have any mutant nucleotides (FIG. 11c). However, in the sequence of the PCR product of the mitochondrial atp1 in these 8 plants, such homoplasmic substitution from the C:G pair to the T:A pair was observed (FIG. 18). On the other hand, in the PCR product sequence of the nuclear genome atp1-like sequence (At2g07698), no substitution was observed in the sequence corresponding to the target window (FIG. 20). These results supported the assumption that the wild-type C:G SNP detected in the whole genome sequencing would be derived from a nuclear atp1-like sequence. Moreover, basically, no nucleotides were substituted in this sequence (FIG. 20), and low-frequency off-target mutations in the sequence (1397CN 24-10 and 12: FIG. 20) can be removed by mating. In any case, no major off-target mutations were detected either in the mitochondrial genome (FIG. 11c), or in the nuclear DNA sequence similar to the target window (FIG. 20).

II-2-4. Complementation of Phenotypes of Ppr Mutants Using mtpTALECD

RNA editing is a feature of the mitochondrial and chloroplast genomes of land plants, in which the specific Cs of RNA molecules after transcription are converted to U. This is mediated by mitochondria-targeted PPR proteins encoded in the nucleus (Small et al., Plant J. 101, 1040-1056, 2020). In order to verify the usefulness of mtpTALECD in the molecular analysis of the mitochondrial genome, two experiments related to RNA editing were carried out. First, the otp87 mutant exhibiting growth retardation was examined. In wild-type plants, the PPR protein OTP87 converts 1178C of the atp1 transcript (C10 in the target window, FIG. 10a) and 27C of the nad7 transcript to U (Hammani et al., J. Biol. Chem. 286, 21361-21371, 2011). Since only the former RNA editing causes an amino acid substitution (S393L), the absence of the amino acid substitution has been proposed to be the cause of the growth retardation of otp87. Thus, whether or not the defect in RNA editing, and further, the growth retardation would be ameliorated by substituting 1178C of atp1 with T, at the DNA level, by mtpTALECD, was examined. One of the mtpTALECD expression vectors, 1397CN (FIG. 10b), was introduced into the nuclear genome of the otp87 mutant. Among the examined 14 T₁ plants, 7 plants grew as well as wild-type plants (FIG. 12 and FIG. 21a). These 7 plants had a homoplasmic substitution from 1178C (C10) to T (or U) at the DNA and RNA levels in the main leaf (FIG. 12 and FIG. 21a). These results demonstrate that the inability to edit 1178C in the atp1 transcript is a cause of the growth retardation of the otp87 mutant.

II-2-5. Recognition of atp1 by OTP87

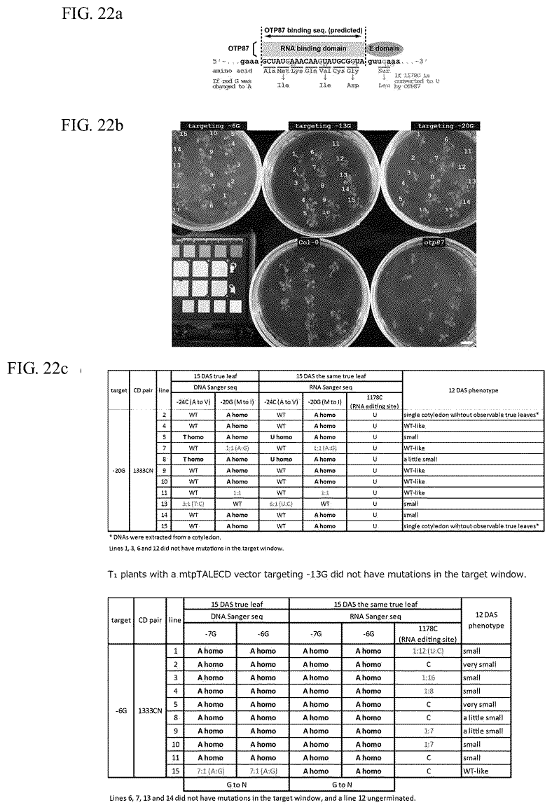

In the second experiment, the atp1 sequence, to which OTP87 is predicted to bind, was examined (Takenaka et al., PloS One 8 e65343, 2013: FIG. 13a and FIG. 22a). The nucleotides to which OTP87, a PLS-type PPR protein is predicted to bind, and the probability thereof, are shown as nucleotide logos in the upper portion of FIG. 13a. These are predicted by the combination of two critical amino acid residues at positions 5 and 35 of each PPR motif [e.g., P, L, S (Takenaka et al., PloS One 8 e65343 2013; Yan et al., Nucleic Acids Res. 47 3728-3738, 2019; Barkan et al., PLOS Genet. 8, e1002910, 2012: and Yagi et al., PloS One 8, e57286, 2013)]. The actual atp1 sequence upstream of the RNA editing site, to which OTP87 is predicted to bind, is shown in the lower portion of FIG. 13a. In the present experiment, in order to examine whether this sequence is necessary for RNA editing and, if so, which nucleotides are involved therein, several C:G pairs in this sequence were substituted with T:A pairs. Three mtpTALECD expression vectors for substituting each of three G₅ at 20, 13, and 6 nucleotides upstream of 1178C with A were constructed (referred to as -20G, -13G, and -6G: FIG. 13a and FIG. 22a). Fifteen T₁ seeds of individual lines (Col-0 background) were seeded, and the DNA and RNA sequences of the seedlings were then analyzed to confirm the pattern of DNA mutation by mtpTALECD and its effect on RNA editing efficiency at 1178C. Although substitution of -13G was not succeeded in the present study, mitochondrial genome mutants with the following 4 allele patterns could be obtained in the predicted OTP87-binding sequence: (i) -24C substituted with T, (ii) -20G substituted with A, (iii) -24C and -20G substituted with T and A, respectively, and (iv) -7G and -6G substituted with A (FIG. 13b). The RNA editing efficiency, which was expressed as Sanger sequencing data of the RT-PCR products of atp1 transcripts, was reduced only in the allele pattern (iv) (FIGS. 13b and c, FIGS. 22a and c, and FIG. 23). These results demonstrate that at least one or two nucleotides of the predicted OTP87-binding sequence actually have an influence on the efficiency of RNA editing, and that -7G and/or -6G are necessary for editing 1178C, and probably, for recognizing and binding to atp1 transcripts. The results also demonstrate that, although -24C and -20G are substituted with U and A, respectively, this case does not have an influence on these activities (at least, does not have a great influence).

### III. Editing of Nuclear Genome

### III-1. Materials and Methods

### III-1-1. Plant Materials, Cultivation Conditions, Transformation, and Screening of Transformant

Arabidopsis thaliana Col-0 and transformants were cultivated at 22° C. under long day conditions (a light period of 16 hours, and a dark period of 8 hours). The Col-0 seeds were seeded on a ½ MS-Agar plate (Non Patent Literature 7). Seedlings with 2 to 3 weeks old were transferred to Jiffy-7 (Jiffy Products International), and were then infected with Agrobacterium. Mature plants of Col-0 were transformed by the floral dip method (Clough et al., The Plant Journal 16, 735-743, 1998.) The obtained T₁ generation was analyzed.

### III-1-2. Designing of TALE-Binding Sequences and Construction of Vectors

Based on the construct of ptpTALECD (Nakazato et al., Nature Plants 7, 906-913, 2021), the chloroplast transition signal (PTP) was substituted with the SV40 nuclear localization signal (SV40NLS) to produce nTALECD. Target sequences were designed for the purpose of introducing stop codons or amino acid substitutions predicted to have a great influence on gene functions into two sites of each of three target loci, AtCYO1, AtPKT3, and AtMSH1, and a total of 6 constructs of nTALECD expression vectors corresponding to individual target sequences were produced, and were then transformed into Col-0 through infection with Agrobacterium by the floral dip method.

### III-1-3. Genotyping of T₁ Plants

PCR for Sanger sequencing was performed employing KOD One PCR Master Mix (Toyobo Co., Ltd.), using DNA roughly extracted from true leaves or cotyledons, according to standard protocols. Nucleic acid templates used in the PCR for Sanger sequencing were extracted using the Maxwell RSC Plant RNA Kit (Promega), without using DNase I included therewith. DNA in the extracted nucleic acids was decomposed with Deoxyribonuclease (RT Grade) for Heat Stop (Nippon Gene) to prepare RNA templates for RT-PCR. The RT-PCR was performed using PrimeScript™ II High Fidelity One Step RT-PCR Kit (TaKaRa). A portion of the mtpTALECD reading frame was amplified with primers, and a transformant was identified. Sequences around the target window of mitochondrial DNA and cDNA and their homologous sequences in the nuclear DNA were amplified. The purified PCR products were read by Sanger sequencing, and the data were analyzed by Geneious Prime (v. 2021. 2.2).

### III-1-4. Image Processing

The photographs of plants were taken with a digital camera (OLYMPUS OM-D E-M5) and were then processed with Adobe Photoshop 2021.

### III-2. Results

### III-2-1. Targeted Single Nucleotide Substitution of CYO1

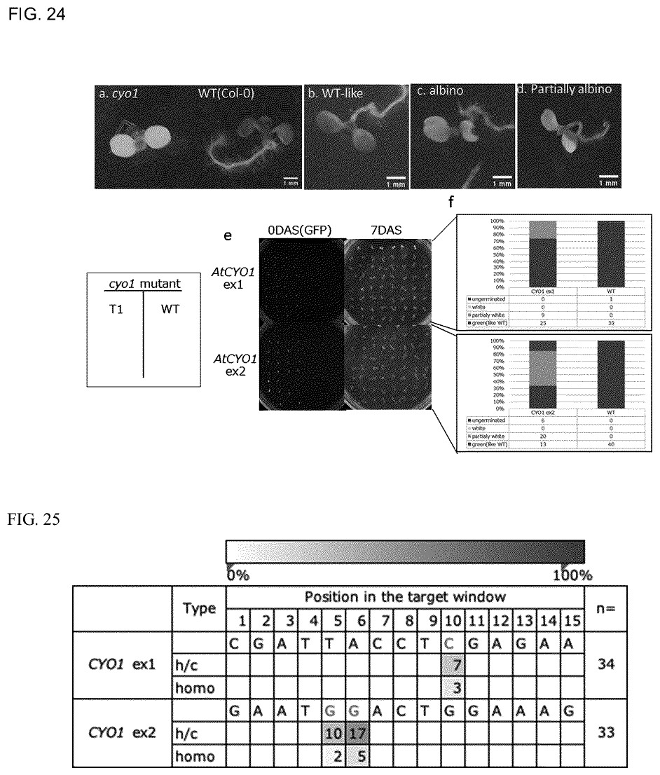

Representative examples of 11 DAS cyo1 mutant and wild type (FIG. 24a) and phenotypes (FIGS. 24b-d) of 7 DAS cotyledons of an nTALECD-introduced T₁ transformant are shown in FIG. 25. The cyo1 mutant shows a phenotype in which only the cotyledons become albino.

Since the cyo1 loss-of-function mutation is a recessive inheritance, it is suggested that the loss-of-function mutation has been introduced into many of T₁ plants, entirely (FIG. 24c) or partially (FIG. 24d), in a biallelic or homozygous mode.

The nucleotide sequence in the target sequence of CYO1 was sequenced by the Sanger method. As a result, it was confirmed that the nucleotide substitution of specific C in the nucleotide sequence occurred at a high efficiency (>40%), and that biallelic/homozygous mutants can be easily obtained in the T₁ generation (FIG. 25).

### III-2-2. Targeted Single Nucleotide Substitution of PKT31 and MSH1

Subsequently, PKT31 and MSH1 were selected as target sequences different from CYO1, and the nucleotide sequences in the target windows of both alleles were sequenced by the Sanger method.

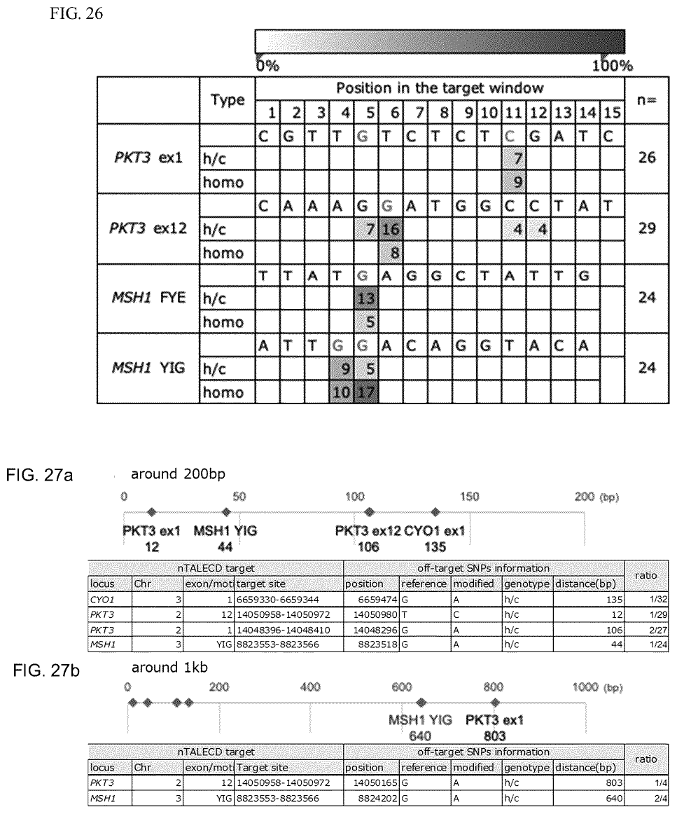

As a result, it was confirmed that the nucleotides C10 and C11 or G₄ to G₆ were edited (FIG. 26). Accordingly, it became clear that single nucleotide editing can be stably carried out on target sequences other than CYO1, and that targeted single nucleotide editing biallelic/homozygous mutants can be easily obtained in all of these target sequences in the T₁ generation.

### III-2-3. Off-Target Editing Around the Target Window

Studies were conducted regarding the degree of occurring the editing of nucleotides other than the target nucleotide, namely, the degree of off-target editing, when single nucleotide substitutions are carried out using the method of the present invention.

As a result, although off-target nucleotide substitutions occurred (TC→TT in all cases), the frequency thereof was low, and indels (insertion and/or deletion of the nucleotide sequence) were not observed around the target sequence (FIG. 27).

### INDUSTRIAL APPLICABILITY

By using the method of the present invention, single nucleotide editing of plant genomes (a nuclear genome, a plastid genome, and a mitochondrial genome) becomes possible. Therefore, plants modified by using the method of the present invention are expected to contribute to the enhancement of food production and the improvement of biofuel production. etc.

1. A method for editing a plant genomic DNA, comprising converting a target nucleotide on the genomic DNA to another nucleotide.

2. The method according to claim 1, wherein the conversion is carried out with cytidine deaminase.

3. The method according to claim 2, wherein the cytidine deaminase is a protein described in the following (a) or (b):
(a) a protein comprising the amino acid sequence as set forth in SEQ ID NO: 35; or
(b) a protein comprising an amino acid sequence having a sequence identity of 90% or more to the amino acid sequence as set forth in SEQ ID NO: 35, and having cytidine deaminase activity.

4. The method according to claim 3, wherein an N-terminal portion of the cytidine deaminase and another portion are each fused with a different transcription activator-like effector (TALE).

5. The method according to claim 3, wherein the conversion comprises introducing a DNA encoding a fusion protein comprising a part of or the entire cytidine deaminase and TALE, to which a nuclear localization signal peptide, a plastid localization signal peptide or a mitochondrial localization signal peptide is added, into a nuclear genome in a plant cell, and then allowing the signal peptide-added fusion protein to express in the plant cell.

6. A plant genome, comprising a plant genomic DNA edited by the method according to claim 1.

7. A plant cell, comprising the plant genome according to claim 6.

8. A seed or a plant, comprising the plant cell according to claim 7.

9. A method for producing a plant having an edited plant genome, the method comprising
editing a plant genome by the method for editing a plant genomic DNA according to claim 1.

10. A plant genome, comprising a plant genomic DNA edited by the method according to claim 5.

11. A plant cell, comprising the plant genome according to claim 10.

12. A seed or a plant, comprising the plant cell according to claim 11.

13. A method for producing a plant having an edited plant genome, the method comprising
editing a plant genome by the method for editing a plant genomic DNA according to claim 5.

14. The method according to claim 4, wherein the conversion comprises introducing a DNA encoding a fusion protein comprising a part of or the entire cytidine deaminase and TALE, to which a nuclear localization signal peptide, a plastid localization signal peptide or a mitochondrial localization signal peptide is added, into a nuclear genome in a plant cell, and then allowing the signal peptide-added fusion protein to express in the plant cell.

15. A plant genome, comprising a plant genomic DNA edited by the method according to claim 14.

16. A plant cell, comprising the plant genome according to claim 15.

17. A seed or a plant, comprising the plant cell according to claim 16.

18. A method for producing a plant having an edited plant genome, the method comprising
editing a plant genome by the method for editing a plant genomic DNA according to claim 14.
